(19) United States
(12) Patent Application Publication
(10) Pub. No.: US 2025/0268916 A1
(43) Pub. Date: Aug. 28, 2025

(54) METHOD OF TREATING CNS DISORDERS WITH NEUROSTEROIDS AND GABAERGIC COMPOUNDS

(71) Applicants: SAGE THERAPEUTICS, INC., Cambridge, MA (US); TRUSTEES OF TUFTS COLLEGE, Medford, MA (US)

(72) Inventors: Michael Andrew Ackley, Sudbury, MA (US); James J. Doherty, Bedford, MA (US); Stephen James Moss, Dover, MA (US); Manasa Lakshmi Parakala, Medford, MA (US); Paul A. Davies, Roslindale, MA (US)

(21) Appl. No.: 18/826,944

(22) Filed: Sep. 6, 2024

Related U.S. Application Data
(63) Continuation of application No. 17/867,279, filed on Jul. 18, 2022, now abandoned.
Application No. 17/867,279 is a continuation of application No. 16/755,307, filed on Apr. 10, 2020, now abandoned.
(60) Provisional application No. 62/571,703, filed on Oct. 12, 2017.

(51) Int. Cl. A61K 31/57; A61K 45/06; A61P 25/00

(52) U.S. Cl. CPC A61K 31/57; A61K 45/06; A61P 25/00

(57) ABSTRACT

Provided herein are methods, therapeutic agents and composition for treating a CNS-related disorder.

### FIELD OF THE INVENTION

The present invention relates to methods and therapeutic agents for treating or preventing CNS-related disorders.

### BACKGROUND OF THE INVENTION

Brain excitability is defined as the level of arousal of an animal, a continuum that ranges from coma to convulsions, and is regulated by various neurotransmitters. In general, neurotransmitters are responsible for regulating the conductance of ions across neuronal membranes. Gamma-aminobutyric acid (GABA) is a major inhibitory neurotransmitter in the mammalian central nervous system (CNS). GABAergic inhibition refers to GABA-mediated neurotransmission which is inhibitory to mature neurons in vertebrates. Bernard C et al., Epilepsia (2000) 41(S6):S90-S95). Activation of GABA receptors by GABA causes hyperpolarization of neuronal membranes and a resultant inhibition of neurotransmitter release, thereby reducing brain excitability.

GABAergic inhibition is implicated in various CNS-related disorders, including but not limited to psychiatric and neurological conditions associated with impaired neuronal excitability, such as rapid mood changes, anxiety, stress response and epilepsy.

New and improved methods, therapeutic agents and compositions capable of modulating GABAergic inhibition are needed for the prevention and treatment of these CNS-related disorders. The methods, therapeutic agents and compositions described herein are directed towards this end.

### SUMMARY OF THE INVENTION

The present invention is based, at least in part, on the surprising discovery that some membrane progesterone receptor (mPR) agonists modulate GABAergic inhibition, and are useful for treating CNS-related disorders. The modulation effect of these candidate GABAergic inhibitors can be allosteric, metabotropic, or both.

In one aspect, provided herein is a method for treating a CNS-related condition or disorder in a subject in need thereof, the method comprising administering to the subject a membrane progesterone receptor (mPR) agonist, wherein the mPR agonist is not progesterone, 5α-DHP, allopregnanolone or testosterone. In some embodiments, the mPR agonist is also a GABAergic modulator. In some embodiments, the mPR agonist is not a GABAergic modulator.

In some embodiments, the invention relates to method for treating a CNS-related condition or disorder in a subject in need thereof, comprising administering to the subject a) a membrane progesterone receptor (mPR) agonist; and b) a GABAergic modulator.

In another aspect, the invention provides a membrane progesterone receptor (mPR) agonist for use in treating a CNS-related condition or disorder in a subject, wherein the mPR agonist is not progesterone, 5α-DHP, allopregnanolone or testosterone. In yet another aspect the invention provides a membrane progesterone receptor (mPR) agonist and a GABAergic modulator for use in treating a CNS-related condition or disorder in a subject.

In another aspect, the invention provides the use of a membrane progesterone receptor (mPR) agonist in the manufacture of a medicament for treating a CNS-related condition or disorder. In yet another aspect the invention provides the use of a membrane progesterone receptor (mPR) agonist and a GABAergic modulator in the preparation of a medicament for treating a CNS-related condition or disorder.

In some embodiments, the GABAergic modulator increases GABAergic inhibition in a cell through modulating intracellular trafficking of GABA receptors.

In some embodiments, the GABAergic modulator increases a membrane-associated amount of at least one GABA receptor subunit.

In some embodiments, the GABAergic modulator increases the membrane-associated amount of the at least one GABA receptor subunit by (1) increasing an amount of the at least one GABA receptor subunit that is located on the cell membrane; (2) increasing an amount of the at least one GABA receptor subunit that is incorporated into a GABA receptor; (3) increasing an ratio between a membrane-associated amount of the at least one GABA receptor subunit and a soluble amount of the at least one GABA receptor subunit; (4) reducing a rate of endocytosis of membrane GABA receptors, or any combination of (1)-(4).

In some embodiments, the GABAergic modulator increases expression of at least one GABA receptor subunit in the cell.

In some embodiments, the GABAergic modulator increases phosphorylation of at least one GABA receptor subunit in the cell.

In some embodiments, the phosphorylation is protein kinase C (PKC)-mediated phosphorylation.

In some embodiments, phosphorylation of an α4 GABA subunit is increased. In some embodiments, phosphorylation of a β3 GABA subunit is increased.

In some embodiments, the phosphorylation occurs at S408/409 of the β3 subunit.

In some embodiments, the at least one GABA receptor subunit is selected from an α1 subunit, a β2 subunit, a γ2 subunit, an α4 subunit, a β3 subunit, and a δ subunit, and any combination thereof.

In some embodiments, the at least one GABA receptor subunit comprises a combination of α1β2γ2 subunits or a combination of α4β3δ subunits.

In some embodiments, the GABA receptor is selected from a synaptic GABA receptor, an extrasynaptic GABA receptor, and a combination thereof.

In some embodiments, the synaptic GABA receptor comprises one or more subunits selected from an al subunit, a β2 subunit, and a γ2 subunit.

In some embodiments, the extrasynaptic GABA receptor comprises one or more subunits selected from an α4 subunit, a β3 subunit, and a δ subunit.

In some embodiments, the GABAergic modulator increases GABAergic inhibition through potentiating GABA receptors in a cell.

In some embodiments, the GABAergic modulator increases the GABAergic current of the cell.

In some embodiments, the GABAergic current is a tonic current and/or a spontaneous inhibitory post-synpatic current (sIPSC).

In some embodiments, the GABAergic modulator increases (1) an average amplitude of the tonic current; (2) an average current density of the tonic current; (3) an average amplitude of the sIPSC; (4) an average decay time of the sIPSC, or any combination of (1)-(4).

In some embodiments, the mPR agonist is a natural or synthetic neuroactive steroid. In some embodiments, the mPR agonist is a progesterone analog.

In some embodiments, the GABA receptor is GABA_{A} receptor.

In some embodiments, the mPR agonist activates a mPR signaling pathway in a cell.

In some embodiments, upon activation of the mPR signaling pathway, protein kinase C (PKC) activity increases. In some embodiments, upon activation of the mPR signaling pathway, the level of cellular cAMP reduces. In some embodiments, upon activation of the mPR signaling pathway, the level of GABA-independent neural inhibition in the subject increases.

In some embodiments, the cell is a brain cell. In some embodiments, the cell is a neuron.

In some embodiments, the CNS-related condition or disorder is a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, and/or a neurodegenerative disease and/or disorder.

In some embodiments, the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus.

In some embodiments, the mPR agonist and/or GABAergic modulator is administered in an amount sufficient to beneficially alter the CNS-related condition or disorder in said subject.

In some embodiments, the mPR agonist and/or GABAergic modulator is administered in an amount sufficient to beneficially alter brain excitability in said subject.

In some embodiments, the mPR agonist and/or the GABAergic modulator is administered orally, parenterally, intradermally, intrathecally, intramuscularly, subcutaneously, or transdermally.

### BRIEF DESCRIPTION OF THE DRAWINGS

FIG. 1 shows NASs (neuroactive steroids) allosterically modulate DGGC (dentate gyrus granule cells) tonic currents. Panel (A) is a scheme demonstrating experimental protocol. Panel (B) is an example of current recordings from DGGCs showing modulation of tonic currents by acutely applied NASs (left panel). Bars above current recordings represent application of NAS and picrotoxin. Bar graphs summarizing the average tonic current (mean±SEM) before and during acute exposure with ALLO, SGE-516, or ganaxolone (right panel). *=significantly different to control (p=0.004; t-test), n=4-12 cells.

FIG. 2 shows allosteric modulation of phasic currents by acutely applied NASs. Recordings of sIPSCs from DGGCs before and during acute exposure to 100 nM ALLO, 100 nM SGE-516, or 100 nM ganaxolone for 10 min. Example of current recordings from DGGCs showing phasic currents before and during acutely applied NASs (left panel). Bar graphs summarizing the effects of acute exposure of ALLO, SGE-516, or ganaxolone on the amplitude and decay of sIPSCs (right panel). ***p=0.01, **p=0.03, *p=0.04, paired t-test, n=5-7 cells.

FIG. 3. shows NAS-mediated metabotropic enhancement of tonic inhibitory current in DGGC neurons. Panel (A) is a scheme demonstrating the experimental protocol. Left panel B, C, D show example tonic currents from slices following exposures to vehicle (control) or 100 nM ALLO (B), 100 nM SGE-516 (C), or 1 μM ganaxolone (D) for 15 min. No change in tonic current was observed in slices pre-incubated for 15 min with GFX followed by ALLO, or SGE-516. Bar above current represents application of picrotoxin (100 mM). Right panel B, C, D are bar graph showing that average tonic current was significantly enhanced following exposure to different concentrations of ALLO and SGE-516. No significant change in tonic current was observed following exposure to 1 μM ganaxolone for 15 min. In all panels *=significantly different to control (p<0.05; un-paired t-test, n=4-12 cells).

FIG. 4 shows glycine receptors do not contribute to tonic current in DGGCs. Hippocampal slices were incubated for 15 min with 100 nM ALLO or vehicle dissolved in ACSF then transferred to the recording chamber and washed for 30-60 min with NAS-free ACSF before recordings were started. Tonic current was measured by applying 100 μM picrotoxin in the absence or presence of the glycine receptor, strychnine (100 nM). Exposure to ALLO caused a significant increase in tonic current. Addition of strychnine did not alter the tonic current measured with picrotoxin. *p=0.01, unpaired t-test, n=4-12 neurons.

FIG. 5 shows sIPSC amplitude and decay was largely unchanged following exposure to NASs. Panel (A) shows IPSC recordings made from DGGCS in hippocampal slices from p21-35 C57 mice exposed to vehicle (control, n=6 neurons from 3 mice); panel (B) shows IPSC recordings made from DGGCS in hippocampal slices from p21-35 C57 mice exposed to 100 nM ALLO (n=4 neurons from, 2 mice); panel (C) shows IPSC recordings made from DGGCS in hippocampal slices from p21-35 C57 mice exposed to 100 nM SGE-516 (n=5 neurons from 2 mice); and panel (D) shows IPSC recordings made from DGGCS in hippocampal slices from p21-35 C57 mice exposed to 1 μM ganaxolone (n=5 neurons from 2 mice); for 15 min and then washed for >30 min prior to measurement of sIPSCs. Bar graphs show average sIPSC decay and amplitude. Only sIPSC amplitude was significantly enhanced following exposure to 100 nM SGE-516 but GFX (n=5 neurons from 2 mice) significantly reduced SGE-516 enhancement. *=significantly different to control (p<0.05; unpaired t-test).

FIG. 6. shows NAS exposure increases phosphorylation and surface expression of β3 subunits. Panel (A) shows exposure to 100 nM of the NASs, ALLO or SGE-516, for 20 min increases β3 S408/409 phosphorylation in acute hippocampal slices. Panel (B) shows the ratio of p-β3/T-β3 normalized to those in control (100%). Asterisks represent a significant difference from control (ALLO: p<0.01, Student's t-test, n=10 slices, from 10 mice; SGE-516: p<0.05, Student's t-test, n=4 slices, from 4 mice). Panel (C) shows exposure to 100 nM ALLO or SGE-516 for 20 min increases GABA_{A}-β3-containing receptors at the plasma membrane in acute hippocampal slices. Panel (D) shows the ratio of surface β3/T-β3 normalized to cell surface levels in control treated slices (100%). Asterisks represent a significant difference from control (ALLO: p<0.05, Student's t-test, n=8 slices; SGE-516: p<0.05, Student's t-test, n=4 slices).

FIG. 7 shows neurosteroids increase phosphorylation of GABA_{A}Rs and their cell surface stability. Panel (A) shows immunoblotting experiments of hippocampal slices treated with vehicle (Con), or SGE-516 for 20 min. The ratio of pS408/9/β3 immunoreactivity was normalized to control slices (100%=the line). Panel (B) shows the results of affinity purified pS443 used to immunoblot varying concentrations of the immunizing phosphor-peptide (PP). pS443 was used to immunoblot extracts of hippocampal slices treated without preadsorption (0), preadsorbed with the dephosphorylated (DP), or phosphorylated antigen (PP). Panel (C) shows immunoblotting experiments of hippocampal slices treated with vehicle (Con) or 100 SGE-516 for 5 min and then immunoblotted with pS443 and α4 antibodies as indicated. Panel (D) shows immunoblotting experiments of hippocampal slices treated as outlined above. Surface(S) and total (T) fractions were immunoblotted with α4 and β3 subunit antibodies. The ratio of S/T immunoreactivity and was normalized to control slices (100%=the line). Panel (E) shows the effect of diazepam (DZ) on cell surface stability of the β3 subunit. Panel (F) shows immunoblotting experiments of hippocampal extracts from C57/β16 mice injected with SGE-516 (5 mg/kg IP), or vehicle. SDS-soluble hippocampal extracts were immunoblotted with pS443, α4, pS408/9, or β3 subunit antibodies. In all panels: data represent mean±s.e.m.*=significantly different to control p<0.05 (t-test; n=7 mice).

FIG. 8 shows mutation of S408/9 in the 3 blocks the ability of SGE-516 to induce sustained effects on GABAergic inhibition. Panel (A) shows an experimental protocol used to examine the metabotropic effects of NASs on GABAergic currents. Panel (B) shows the sustained effects of SGE-516 on tonic currents measured in DGGCs from WT and S408/9A mice. Tonic current density was then compared between slices exposed to vehicle or SGE-516. *=significantly different control, p<0.05 (t-test; n=8 mice).

FIG. 9 shows that mutation of S408/9 in the β3 blocks the effects of SGE-516 on the cell surface levels of GABA_{A}Rs. Left panel (wide type) and right panel (S408/409A mutant) shows immunoblotting experiments of hippocampal slices treated as outlined above. Surface(S) and total (T) fractions were immunoblotted with α4 and β3 subunit antibodies. The ratio of S/T immunoreactivity and was normalized to control slices (100%=the line).

FIG. 10 shows measurements in the decay time of mIPSc in wild type (left panel) and S408/409A mutant (right panel) mice before and after treatment of ALLO.

FIGS. 11A and B show diagrams representing the protocols used to induced pharmacoresistant seizures in WT and S408/9A mice using kainate as measured using EEG recording.

FIGS. 12A and B show the ability of diazepam, SGE-516, THDOC in modifying seizure activity in S408/9 mice using EEG recording.

FIG. 13 shows % change in seizure power 10 minutes after treatment by diazepam, SGE-516, THDOC in wild type and S408/9A mutant mice.

FIG. 14 shows the diversity in ability of neuroactive steroids in modulating GABA receptor trafficking.

FIG. 15 shows that allopregnanolone (ALLO) and progesterone (P4) increase S408/9 phosphorylation in GT1-7 cells. Panel (A) upper section shows quantitative PCR analysis showing the enrichment of the mPRα mRNA in GT1-7 cells (taken from Thomas and Peng 2012). Lower section shows immunoblotting of 10 and 15 μg of SDS-soluble extracts from GT1-7 cells with an mPRα specific antibody. Panel (B) shows GT1-7 immunoblotting of cells treated with 100 nM ALLO or P4 for 15 min and immunoblotted with pS408/9, β3 and actin antibodies. The ratio of pS408/9/β3 subunit immunoreactivity were then normalized to levels seen in vehicle treated to controls, n=6.

FIG. 16 shows ALLO and ORG OD 02-0 induced sustained increases in GABA-evoked currents recorded from GT1-7 cells.

FIG. 17 shows that ORG OD 02-0 (ORG) compound does not acutely modulate of the function of GABA_{A}Rs composed of α4β3 subunits. Upper panel shows sample traces of whole cell recording of GABA-induced currents (I_{GABA}) from cells treated with rapidly applied GABA (G), GABA and 100 nM ALLO (G&ALLO), or GABA and 100 mM ORG OD 02-0 (G&ORG). Lower panel shows the quantitation of percentage enhancement of I_{GABA} induced by the treatment.

FIG. 18 shows that P4 and ORG OD 02-0 regulated S408/9 phosphorylation in hippocampal slices. Panel (D) shows immunoblotting experiments of hippocampal slices were treated with 100 nM ALLO or P4 (progesterone), and S408/9 phosphorylation was then determined as detailed above, n=4 slices. Panel (E) shows immunoblotting experiments of Hippocampal slices were treated with 100 nM ORG OD 02-0, and S408/9 phosphorylation was examined using immunoblotting. In all panels; *=significantly different to control p<0.05 (one way ANOVA with Dunnet's multiple comparisons post-hoc test).

FIG. 19 shows dosage dependent effect of P4 and ORG OD 02-0 in modulating GABAergic tonic current. Left panel shows dosage-dependent effect of P4 and Org OD 02-0 in modulating amplitude of tonic current. Right panel shows dosage-dependent effect of P4 and ORG OD 02-0 in modulating density of tonic current.

FIG. 20 shows the mechanism of the mPR agonist-induced sustained elevations in GABAergic inhibition by promoting mPR-dependent phosphorylation of GABA_{A}Rs. Particularly, an mPR agonist such as a neuroactive steroids activates mPRs, which further activates protein kinase C (PKC), resulting in phosphorylation of GABA_{A}Rs on residues that include S408/9 in the β3 GABA receptor subunit. Enhanced phosphorylation for example at S408/9 results in enhanced trafficking of GABA_{A}Rs, an event that leads to a higher membrane density of GABA_{A}Rs, as well as a sustained increase in the efficacies of GABAergic phasic and tonic GABAergic inhibition.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Each embodiment of the invention described herein may be taken alone or in combination with one or more other embodiments of the invention.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Throughout this specification, the word “comprise” or variations such as “comprises” or “comprising” will be understood to imply the inclusion of a stated integer or groups of integers but not the exclusion of any other integer or group of integers. In each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation.

The term “including” is used to mean “including but not limited to.” “Including” and “including but not limited to” are used interchangeably.

The singular forms “a,” “an,” and “the” include the plurals unless the context clearly dictates otherwise.

In order to further define the invention, the following terms and definitions are provided herein.

### Definitions

The term “GABAergic inhibition” refers to GABA (γ-Aminobutyric acid) mediated neurotransmission which is inhibitory to mature neurons in vertebrates. Bernard C et al., Epilepsia (2000) 41(S6):S90-S95).

GABA (γ-Aminobutyric acid) is a major inhibitory neurotransmitter in the spinal cord dorsal horn. GABA mediates both phasic and tonic inhibitory neurotransmission in the CNS through GABA_{A} receptors (GABA_{A}R_{S}). Particularly, GABA is released from presynaptic terminals of inhibitory neurons. Upon binding to GABA_{A} receptors at postsynaptic membrane, GABA elicits inhibitory postsynaptic currents (IPSCs). IPSCs provide phasic inhibition in neuronal network and are important for information processing. In addition to its action at synaptic sites, recent studies in several brain regions of matured animals have indicated that low concentrations of ambient GABA can activate high affinity GABA_{A} receptors that are expressed at extrasynaptic sites to elicit a sustained inhibitory current. Ataka et al. Mol Pain. 2006; 2:36. The term “tonic inhibitory current” or “tonic current” has been used to describe this sustained inhibitory current. Functionally, tonic GABAergic inhibition has been shown to control neuronal excitability in the brain.

GABA_{A} receptors (GABA_{A}Rs) are heteropentameric ligand-gated ion channels that selectively permit the influx of Cl⁻ and HCO₃⁻ ions to decrease membrane excitability. Extremely heterologous with at least nineteen known subunit genes, GABA_{A} receptors mediate the majority of fast synaptic inhibition. GABA_{B} receptors (GABA_{B}Rs) are metabotropic G protein-coupled heterodimers of GABA-β1 and GABA-β2. They are expressed on both the presynaptic and postsynaptic terminals where they inhibit neurotransmitter release and induce cell membrane hyperpolarization, respectively. A third group of receptors was originally classified as GABA_{C} receptors (GABA_{C}Rs). These receptors are now considered as members of the GABA_{A} family.

GABA receptor subunits: GABA_{A}Rs are heteropentamers constructed from α(1-6), β(1-3), γ(1-3), δ, ε(1-3), θ, and/or π subunits. There are thousands of possible subunit combinations, however only relatively few are expressed with any frequency in the mammalian central nervous system. Most GABA_{A}Rs are composed of 2α, 2β and 1γ (or 1δ) subunit. GABA_{A}Rs with different subunit composition have different physiological and pharmacological properties, are differentially expressed throughout the brain, and targeted to different subcellular regions. For instance, receptors composed of α(1, 2, 3 or 5) subunits together with β and γ subunits are largely synaptically located and mediate the majority of phasic inhibition in the brain (with the notable exception of extrasynaptically-localized α5-containing receptors). In contrast, those composed of α(4/6)βδ subunits form a specialized population of predominantly extrasynaptic receptor subtypes that mediate tonic inhibition. In addition, GABA_{A}Rs at presynaptic sites also exist. Jacob et al. Nat Rev Neurosci. 2008 May; 9(5):331 343; Brickley S G, et al. Neuron 73(1):23-34.

Intracellular trafficking of GABA_{A} receptor: GABA_{A}Rs go through an intracellular trafficking cycle which begins with the assembly of the receptors in the endoplasmic reticulum (ER). After assembly in the ER, transport-competent GABA_{A}Rs are trafficked to the Golgi apparatus and segregated into vesicles for transport to, and insertion into, the plasma membrane, where they are able to access inhibitory postsynaptic specializations or extrasynaptic sites, depending on subunit composition. Membrane-associated GABA_{A}Rs undergo extensive endocytosis in both heterologous and neuronal systems. For example, approximately 25% of β3-containing cell surface GABA_{A}Rs being internalized within 30 minutes. Once endocytosed, most internalized GABA_{A}Rs recycle back to the plasma membrane over short time frames; however over longer time periods they are targeted for lysosomal degradation. Various protein factors are known to play a role in the intracellular trafficking of GABA_{A}Rs, including but are not limited to ubiquitin-proteasome system (UPS), ubiquitin-like proteins Plic-1 and Plic-2, N-ethylmaleimide-sensitive factor (NSF), GABA receptor-associated protein (GABARAP), Golgi-specific DHHC zinc finger domain protein (GODZ), BIG2, GRIF/TRAK proteins, Gephyrin, an ERM (ezrin, radixin, moesin)-family member protein, clathrin adaptor protein 2 (AP2) complex, and Huntingtin associated protein-1 (HAP1). Jacob et al. Nat Rev Neurosci. 2008 May; 9(5):331-343.

The term “modulation” of an activity or physical state of a protein as used herein means increasing or decreasing an activity of that protein or a property of the protein's physical state resulting from contacting a test or candidate compound to a suitable test system. The modulation may be relative to another activity or property of a different protein, to the same protein in the basal state or subsequent to external stimulation, including contacting GABA to the test system prior to contacting of the testing agent, or relative to the change in activity or property from contacting the test system with vehicle or reference compound.

The term “modulator” of an activity or physical state of a protein as used herein refers to an agent or a composition comprising that agent which acts to increase or decrease the activity of that protein or property of the protein's physical state. For example, GABAergic modulators increase or decrease GABAergic inhibition in either an in vivo or in vitro setting.

The term “GABAergic modulator” as used herein refers to an agent which, upon being introduced to a test system, acts to modulate GABAergic inhibition via one or more mechanisms. Effect of a GABAergic modulator can be (1) allosteric, (2) metabotropic, or (3) both.

The term “allosteric modulation” as used herein refers to the process of modulating a receptor by the binding of allosteric modulators at a site (i.e., regulatory site) other than that of the endogenous ligand (orthosteric ligand) of the receptor and enhancing or inhibiting the effects of the endogenous ligand. An allosteric modulator generally acts by causing a conformational change in a receptor molecule, which results in a change in the binding affinity of the ligand. Thus, an allosteric ligand (or modulator) modulates activation of a receptor by a primary “ligand” and can adjust the intensity of the receptor's activation. The effect of allosteric modulation is usually acute, arising immediately after exposing the test system to the allosteric modulator, and disappearing soon after the allosteric modulator is removed from the test system.

A “positive allosteric modulator (PAM)” enhances the effect of the endogenous ligand. For example, a PAM of GABA receptors typically interacts with the GABA receptor at a site different from the binding site of the orthosteric ligand-GABA, and enhances GABAergic inhibition. In some embodiments, allosteric modulation effect arises immediately after the test system is exposed to the modulator, and stops quickly after the modulator is removed from the system.

The term “metabotropic modulation” as used herein refers to the process of modulating a GABA receptor activity through signal transduction mechanisms. Metabotropic modulation can be sustained for a period of time after the metabotropic modulator has been removed from the test system.

Membrane progesterone receptors (mPRs) are G protein-coupled receptors belonging to the progestin and adipoQ receptor family (PAQR) that mediate a variety of rapid, cell surface-initiated progesterone action involving activation of intracellular signaling pathways. Human mPRs are classified into the following subtypes: mPRα (encoded by the PAQR7 gene), mPRβ (encoded by the PAQR8 gene), mPRγ (encoded by the PAQR5 gene), mPRδ (encoded by the PAQR6 gene), and mPRϵ (encoded by the PAQR9 gene). Analysis of mPR expression in various human tissues shows variable distribution of mPRα, mPRβ, and mPRγ. All three mPRs, mPRα, mPRβ, and mPRγ are found in human brain, including expression in the spinal cord, cerebral cortex, cerebellum, thalamus, pituitary gland, and caudate nucleus. Dressing et al. Steroids. 2011 January; 76(1-2):11-17. The mPR can also bind to neurosteroids, such as progesterone and allopregnanolone. Thomas P, Pang Y (2012). Neuroendocrinology. 96(2):162-71. Petersen S L, et al. (2013). Frontiers in Neuroscience. 7:164.

The term “signaling pathway” or “signal transduction pathway” as used herein refers to a sequence of biochemical events or the proteins and relay molecules involved in these events that transfer the consequence of a ligand binding event originating externally or internally to a cell or a cell-free system to an effector protein or receptor. The consequence (or signal) from these initial binding events are then transferred to another protein whose catalytic action or its effect on the catalytic action of another downstream protein amplifies the signal, which then may be passed along to yet another protein for further amplification to eventually modulate the activity or phosphorylation state of an effector protein or substrate terminal to the signal transduction cascade.

“Signal transduction node” as used herein refers to a component of a signal transduction pathway capable of having catalytic activity for incoming signal amplification. A signal transduction node may be an effector protein, protein complex, or non-protein component capable of this catalytic activity. The catalytic activity may be dependent upon the phosphorylation states of the effector protein, or one or more protein kinases that act upon them, or activities of effector molecules from other signal transduction pathways.

The term “phosphorylation status” or “phosphorylation state” or “phosphorylation level” as used herein interchangeably refers to the number or pattern of phosphate groups covalently bound to a phospho-protein, such as a phosphorylated GABA receptor subunit, which may be soluble, membrane bound and/or in a protein complex. For example, phosphorylation status may refer to the overall extent of phosphorylation of a collection of proteins for a specified protein complex or to the extent to which specified amino acid residue(s) of a specified protein in collection of such proteins that are capable of being phosphorylated are in fact phosphorylated. Protein Phosphorylation is typically catalyzed by protein kinases. Different protein kinases have different specificity and preference for substrates. For example, protein kinase C (PKC) is a family of protein kinases that are involved in controlling the function of other proteins through phosphorylation of hydroxyl groups of serine and threonine amino acid residues on these proteins.

“Candidate GABA receptor trafficking modulator” as used herein refers to GABAergic modulators that affects the intracellular trafficking cycle of a GABA receptor. The modulation effect can be positive or negative. A positive modulation on the GABA receptor trafficking results in more functional GABA receptors in a test system, thus strengthening GABAergic inhibition. A negative modulation on the GABA receptor trafficking results in less functional GABA receptors in the test system, thus weakening GABAergic inhibition.

“A functional GABA receptor” as used herein refers to fully assembled GABA receptors that have been inserted into a membrane, thereby contributing to the electrical permeability of the membrane under the control of GABA, and other GABAergic modulators.

“Candidate GABA receptor potentiator” as used herein refers to GABAergic modulators that affects electrical permeability of a functional GABA receptor. Modulation by a candidate GABA receptor potentiator may be through the control of the open/close state of the ion channel formed by the GABA receptor. The term “open/close state” of an ion channel including whether the ion channel is open or closed at a given moment, the dimension of the opened channel at a given moment, the frequency of the ion channel becoming open in a given unit time, and/or the time duration of the ion change staying in the open state in a given unit time.

The term “neurosteroids” or “neuroactive steroid (NAS)” as used herein refers to a class of steroids, the natural forms of which are produced by cells of the central or peripheral nervous systems, independently of the steroidogenic activity of the endocrine glands. The neuroactive steroids as used herein can alter neuronal excitability through direct or indirect interaction with ligand-gated ion channels and/or other cell surface receptors. One class of neuroactive steroids are GABAergic modulators. Neuroactive steroid as used herein includes synthetic compounds, such as functional and/or structural analogs of natural neuroactive steroids.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, an “effective amount” of a compound refers to an amount sufficient to elicit a desired biological response. As used herein, an effective amount is sufficient to beneficially alter the CNS-related condition or disorder in a subject (e.g. beneficially altering brain excitability in a subject). As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, the terms “treatment,” “treating,” and the like, generally means the improvement of a disease, disorder or condition, or its symptoms, or lessening the severity of the disease, disorder or condition or its symptoms.

As used herein, and unless otherwise specified, a “pharmaceutical composition” refers to the active ingredient in combination with a pharmaceutically acceptable carrier, e.g. a carrier commonly used in the pharmaceutical industry.

As used herein, the term “active ingredient” refers to the agent or component of a composition that is accountable for the desired therapeutic effect of the composition. In the context of the present disclosure, an active ingredient is included in a pharmaceutical composition or administered to a subject in an amount sufficient to elicit a biological effect of modulating GABAergic inhibition, modulating an activity associated with the mPR mediated pathway, or both.

### Method of Treatment

In one aspect, provided herein are methods for treating a CNS-related disorder or condition in a subject in need thereof. In some embodiments, the method comprises administering to the subject a therapeutic agent according to the present disclosure. In some embodiments, the therapeutic agent to be administered is an mPR agonist. In some embodiments, the therapeutic agent to be administered is not progesterone, 5α-DHP, allopregnanolone or testosterone. In some embodiments, the administered mPR agonist is also a GABAergic modulator. In other embodiment, the administered mPR agonist is not a GABAergic modulator. In some embodiments, where the administered mPR agonist is not a GABAergic modulator, the method further comprises administering to the subject in need thereof a second therapeutic agent that is a GABAergic modulator.

In some embodiments, the method comprises administering to the subject in need thereof a combination of an mPR agonist and a GABAergic modulator. In some embodiments, upon administration to the subject, the GABAergic modulator increases GABAergic inhibition through modulating intracellular trafficking of GABA receptors in a cell of the subject. In some embodiments, upon administration to the subject, the GABAergic modulator increases a membrane-associated amount of at least one GABA receptor or GABA receptor subunit.

In some embodiments, upon administration to the subject, the GABAergic modulator acts to increases the membrane-associated amount of at least one GABA receptor subunit by (1) increasing an amount of the at least one GABA receptor subunit that is located on the cell membrane; (2) increasing an amount of the at least one GABA receptor subunit that is incorporated into a GABA receptor; (3) increasing an ratio between a membrane-associated amount of the at least one GABA receptor subunit and a soluble amount of the at least one GABA receptor subunit; (4) reducing a rate of endocytosis of membrane GABA receptors, or any combination of (1)-(4).

In some embodiments, upon administration to the subject, the GABAergic modulator increases expression of at least one GABA receptor subunit in a cell of the subject.

In some embodiments, upon administration to the subject, the GABAergic modulator increases phosphorylation of at least one GABA receptor subunit in the cell. In some embodiments, the phosphorylation is protein kinase C (PKC)-mediated phosphorylation. In some embodiments, the phosphorylation is Ca²⁺/calmodulin-dependent protein kinase II (CaMKII)-mediated phosphorylation. In some embodiments, upon administration to the subject, the GABAergic modulator increases the phosphorylation of an α4 GABA subunit. In some embodiments, upon administration to the subject, the GABAergic modulator increases the phosphorylation of a β3 GABA subunit. In some embodiments, upon administration to the subject, the GABAergic modulator increases the phosphorylation of the S443 positions of an α4 GABA subunit. In some embodiments, upon administration to the subject, the GABAergic modulator increases the phosphorylation of the S408/409 positions of a β3 GABA subunit. In some embodiments, upon administration to the subject, the GABAergic modulator increases the phosphorylation of a β1 GABA subunit. In some embodiments, upon administration to the subject, the GABAergic modulator increases the phosphorylation of a β2 GABA subunit. In some embodiments, upon administration to the subject, the GABAergic modulator increases the phosphorylation of a γ2 GABA subunit.

In some embodiments, upon administration to the subject, the GABAergic modulator modulates at least one GABA receptor subunit selected from α(1-6), β(1-3), γ(1-3), δ, ε(1-3), θ, and π subunits. In some embodiments, upon administration to the subject, the GABAergic modulator modulates a GABA receptor comprising at least one of α (1, 2, 3 or 5), β and γ subunits. In some embodiments, upon administration to the subject, the GABAergic modulator modulates a GABA receptor comprising at least one GABA receptor subunit selected from α (4/6), β, δ subunits. In some embodiments, upon administration to the subject, the GABAergic modulator modulates a GABA receptor comprises at least one GABA receptor subunit selected from 2α, 2β and 1γ (or 18) GABA receptor subunits. In some embodiments, upon administration to the subject, the GABAergic modulator modulates a GABA receptor comprising at least α1, β2 and γ2 GABA receptor subunits. In some embodiments, upon administration to the subject, the GABAergic modulator modulates a GABA receptor comprising at least α4, β3 and δ GABA receptor subunits.

In some embodiments, upon administration to the subject, the GABAergic modulator modulates a GABA_{A}R. In some embodiments, upon administration to the subject, the GABAergic modulator modulates a synaptic GABA_{A}R. In some embodiments, upon administration to the subject, the GABAergic modulator modulates an extrasynaptic GABA_{A}R. In some embodiments, upon administration to the subject, the GABAergic modulator modulates both synaptic and extrasynaptic GABA_{A}Rs.

In some embodiments, upon administration to the subject, the GABAergic modulator increases GABAergic inhibition through potentiating GABA receptors in a cell of the subject. In some embodiments, upon administration to the subject, the GABAergic modulator increases a GABAergic current of the cell. In some embodiments, the GABAergic current is a tonic current and/or a spontaneous inhibitory post-synpatic current (sIPSC).

In some embodiments, upon administration to the subject, the GABAergic modulator increases (1) an average amplitude of the tonic current; (2) an average current density of the tonic current; (3) an average amplitude of the sIPSC; (4) an average decay time of the sIPSC, or any combination of (1)-(4).

In some embodiments, the method comprises administering to the subject in need thereof a mPR agonist. In some embodiments, the mPR agonist is a natural or synthetic neuroactive steroid. In some embodiments, the mPR agonist is a progesterone analog. In some embodiments, upon administration to the subject, the mPR agonist activates an mPR signaling pathway in a cell of the subject. In some embodiments, upon activation of the mPR signaling pathway, protein kinase C (PKC) activity increases in a cell of the subject. In some embodiments, upon activation of the mPR signaling pathway, the level of cellular CAMP reduces in a cell of the subject. In some embodiments, upon activation of the mPR signaling pathway, the level of cellular cAMP increases in a cell of the subject. In some embodiments, wherein upon activation of the mPR signaling pathway, the level of GABA-independent neural inhibition in the subject increases. In some embodiments, upon activation of the mPR signaling pathway, CaMKII is activated. In some embodiments, upon activation of the mPR signaling pathway, the multi-subunit G protein is activated. In some embodiments, upon activation of the mPR signaling pathway, Gαi subunit of the multi-subunit G protein is activated. In some embodiments, upon activation of the mPR signaling pathway, Gβγ subunit of the multi-subunit G protein is activated. In some embodiments, upon activation of the mPR signaling pathway, PLC is activated. In some embodiments, upon activation of the mPR signaling pathway, PI3K is activated. In some embodiments, upon activation of the mPR signaling pathway, the amount of DAG in a cell of the subject is increased. In some embodiments, upon activation of the mPR signaling pathway, the amount of IP3 in a cell of the subject is increased. In some embodiments, upon activation of the mPR signaling pathway, the amount of mobilized intracellular Ca²⁺ is increased. In some embodiments, upon activation of the mPR signaling pathway, protein kinase A (PKA) is activated. In some embodiments, upon activation of the mPR signaling pathway, proto-oncogene tyrosine-protein kinase Src is activated. In some embodiments, upon activation of the mPR signaling pathway, the mitogen-activated protein kinases (MAPK; also known as extracellular signal-regulated kinase (ERK)) is activated. In some embodiments, upon activation of the mPR signaling pathway, the activity level of cAMP response element-binding protein (CREB) decreases. In some embodiments, upon activation of the mPR signaling pathway, the activity level of cAMP response element-binding protein (CREB) increases. In some embodiments, the cell is a brain cell of the subject. In some embodiments, the cell is a neuron of the subject.

### Therapeutic Agents

In one aspect of the present disclosure, provided herein are therapeutic agents. In some embodiments, the therapeutic agent is an agonist of a membrane progesterone receptor (mPR). In some embodiments, the mPR is mPRα. In some embodiments, the mPR is mPRβ. In some embodiments, the mPR is mPRγ. In some embodiments, the mPR is mPRδ. In some embodiments, the mPR is mPRϵ.

In some embodiments, the therapeutic agent is a neuroactive steroid. In some embodiments, the neuroactive steroid is a natural compound. In some embodiments, the neuroactive steroid is a synthetic compound. In some embodiments, the neuroactive steroid is progesterone, a metabolite or a functional analog thereof. In some embodiments, the neuroactive steroid is a compound selected from the table below:

| Name | Structure |
|---|---|
| Progesterone (P or P4) | 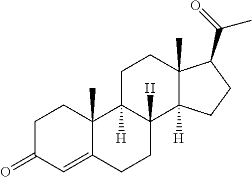 |
| Allopregnanolone (ALLO) | 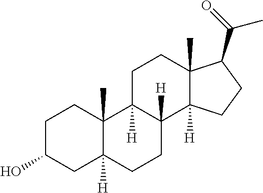 |
| Tetrahydrodeoxycorticosterone (THDOC) | 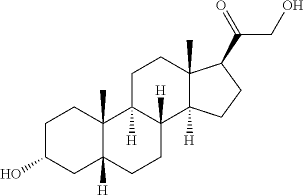 |
| Ganaxolone | 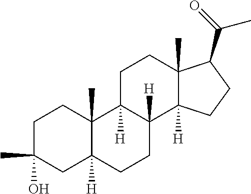 |
| 5α-Dihydroprogesterone (5α-DHP) | 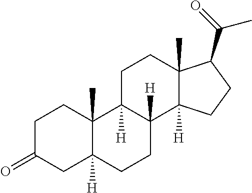 |
| SGE-516 | 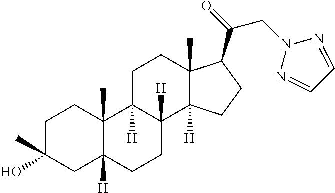 |
| ORG OD 02-0 (ORG OD 020) | 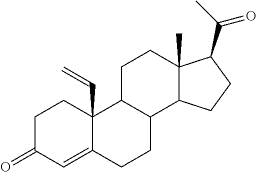 [SMILES: C=C[C@]12CCC(=O)C=C1CCC1C2CC[C@@]2(C)C1CC[C@@H]2C(C)=O] |
| SAGE-217 | 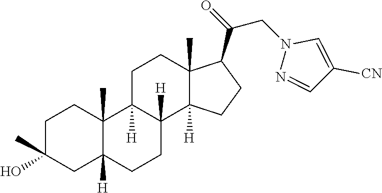 |

In some embodiments, the neuroactive steroid is not progesterone, allopregnanolone (ALLO), 5α-Dihydroprogesterone (5α-DHP) or testosterone.

In some embodiments, the therapeutic agent, upon binding to mPR, activates one or more downstream effector molecule in the mPR mediated signal transduction pathway in the system. In some embodiments, the therapeutic agent activates heterotrimeric G proteins, which consists of three subunits, Gα, Gβ, and Gγ. When a G protein-coupled receptor (GPCR) is activated, Gα dissociates from Gβγ, allowing both subunits to perform their respective downstream signaling effects. In some embodiments, the GABAergic modulator affects the cellular cAMP level. In some embodiments, activated Gαi subunit inhibits the production of CAMP from ATP. In some embodiments, activated Gαi subunit increases the production of CAMP from ATP. In some embodiments, the therapeutic agent inhibits the production of cAMP from ATP. In some embodiments, the therapeutic agent promotes the production of cAMP from ATP. In some embodiments, the therapeutic agent regulates the activity level of cAMP response element-binding protein (CREB). In some embodiments, activated Gβγ subunit activates phospholipase C (PLC). In some embodiments, activated Gβγ subunit activates phosphoinositide 3-kinase (PI3K). In some embodiments, the therapeutic agent activates phosphoinositide 3-kinase (PI3K).

PI3Ks are a family of related intracellular signal transducer enzymes capable of phosphorylating the 3 position hydroxyl group of the inositol ring of phosphatidylinositol (PtdIns). In some embodiments, activated PI3K further activates protein kinase C (PKC). In some embodiments, the therapeutic agent activates kinase C (PKC).

Phospholipase C (PLC) is a class of membrane-associated enzymes that cleave phospholipids just before the phosphate group. In some embodiments, activated PLC further result in production of diacylglycerol (DAG) and inositol 1,4,5-trisphosphate (IP3). In some embodiments, accumulation of DAG and IP3 in the system further stimulates downstream signaling pathways that activate PKC and intracellular Ca²⁺ mobilization. In some embodiments, the therapeutic agent increases cellular DAG and/or IP3 level. In some embodiments, the therapeutic agent increases the amount of mobilized intracellular Ca²⁺.

In some embodiments, immobilization of intracellular Ca²⁺ further activates Ca2+/calmodulin-dependent protein kinase II (CaMKII), which is a serine/threonine-specific protein kinase. In some embodiments, the therapeutic agent activates Ca2+/calmodulin-dependent protein kinase II (CaMKII).

In some embodiments, the therapeutic agent activates one or more protein kinases. In some embodiments, activated protein kinases increases phosphorylation level of one or more GABA receptor subunits. In some embodiments, the increased phosphorylation level of the one or more GABA receptor subunits further increase membrane stability of a GABA receptor comprising the one or more GABA receptor subunits.

In some embodiments, the therapeutic agent activates protein kinase C. In some embodiments, the therapeutic agent activates CaMKII. In some embodiments, the therapeutic agent activates protein kinase A (PKA). In some embodiments, the therapeutic agent activates the proto-oncogene tyrosine-protein kinase Src. In some embodiments, the therapeutic agent activates the mitogen-activated protein kinases (MAPK; also known as extracellular signal-regulated kinase (ERK)).

In some embodiments, the therapeutic agent increases phosphorylation level of a α4 subunit. In some embodiments, the therapeutic agent increases phosphorylation level of a β3 subunit. In some embodiments, the therapeutic agent increases phosphorylation level of a α4 subunit at S443 position. In some embodiments, the therapeutic agent increases phosphorylation level of a β3 subunit at S408/9 positions. In some embodiments, the therapeutic agent increases phosphorylation level of β1 GABA receptor subunit. In some embodiments, the therapeutic agent increases phosphorylation level of β2 GABA receptor subunit. In some embodiments, the therapeutic agent increases phosphorylation level of γ2 GABA receptor subunit.

In some embodiments, the therapeutic agent, upon binding to mPR, upregulates the expression level of at least one GABA receptor subunit. In some embodiments, the upregulated GABA receptor subunit is selected from α(1-6), β(1-3), γ(1-3), δ, ε(1-3), θ, and π subunits. In some embodiments, the upregulated GABA receptor subunit is selected from α (1, 2, 3 or 5), β and γ subunits. In some embodiments, the upregulated GABA receptor subunit is selected from α (4/6), β, δ subunits. In some embodiments, the upregulated GABA receptor subunits comprise 2α, 2β and 1γ (or 18) GABA receptor subunits. In some embodiments, the upregulated GABA receptor subunits comprise α1, β2 and γ2 subunits. In some embodiments, the upregulated GABA receptor subunits comprise α4, β3 and δ subunits.

In some embodiments, the therapeutic agent, upon binding to mPR, upregulates the level of assembly of at least one GABA receptor. In some embodiments, the upregulated GABA receptor is trafficked to and inserted into a synaptic area of a cell membrane. In some embodiments, the upregulated GABA receptor is trafficked to and inserted into an extrasynatpic area of a cell membrane.

In some embodiments, the therapeutic agents are capable of GABAergic modulation. In various embodiments, GABAergic modulation can be via one or more different mechanisms. In some embodiments, the modulation effect of the therapeutic agents can be (1) allosteric, (2) metabotropic or (3) both. As non-limiting examples, in some embodiments, the modulation is through increasing or decreasing the amount of GABA neurotransmitter release in a system. In some embodiments, the modulation is through increasing or decreasing the amount of functional GABA receptors present in a system. In some embodiments, the modulation is through potentiating or inhibiting GABA receptors in the system. As used herein, the system can be in an in vivo or in vitro setting, such as but not limited to a subject, a tissue extracted from the subject, a cell isolated or cultured, or a cell-based system.

In some embodiments, a metabotropic modulation results in a change in the amount of functional GABA receptors in the system. In some embodiments, a positive metabotropic GABAergic modulator acts to increase the overall amount of GABA receptors that are correctly assembled and inserted into cell membrane. In some embodiments, the positive metabotropic GABAergic modulator is a GABA receptor trafficking modulator.

In some embodiments, the therapeutic agent is a GABA receptor trafficking modulator that increases a membrane-associated amount of a GABA receptor by, for example, (1) increasing the level of expression of one or more GABA receptor subunits constituting the GABA receptor, (2) increasing the level of assembly of constituent GABA receptor subunits into the GABA receptor, (3) accelerating intracellular trafficking of the GABA receptor so that more copies of the receptors are trafficked to and inserted into the cell membrane; (4) increasing membrane stability of inserted copies of the GABA receptor so that the receptors stay functional for a longer period of time before endocytosed and recycled; or any combinations of mechanism (1) to (4).

In some embodiments, the therapeutic agent is a GABA receptor potentiator. An allosteric modulation results in a change of potency of functional GABA receptors in the system. In some embodiments, the therapeutic agent is a positive allosteric metabotropic GABAergic modulator that acts to potentiate an existing functional GABA receptor. In some embodiments, the positive allosteric metabotropic GABAergic modulator is a GABA receptor potentiator.

In some embodiments, the GABA receptor potentiator increases electrical permeability of a GABA receptor by, for example, (1) increasing the frequency of the GABA receptor ion channel becoming open in a given unit time; (2) increasing the time duration of the GABA receptor ion channel stays open in a given unit time; (3) increasing the dimension of an opened GABA receptor ion channel; or any combinations of mechanism (1) to (3).

In some embodiments, the therapeutic agent is a GABAergic modulator which has both an allosteric and a metabotropic effect upon GABAergic inhibition. In some embodiments, a positive GABAergic modulator both increases the amount of functional GABA receptors in the system and potentiating existing GABA receptors. In some embodiments, a positive GABAergic modulator functions to (1) increasing the level of expression of one or more GABA receptor subunits constituting the GABA receptor, (2) increasing the level of assembly of constituent GABA receptor subunits into the GABA receptor, (3) accelerating intracellular trafficking of the GABA receptor so that more copies of the receptors are trafficked to and inserted into the cell membrane; (4) increasing membrane stability of inserted copies of the GABA receptor so that the receptors stay functional for a longer period of time before endocytosed and recycled; (5) increasing the frequency of the GABA receptor ion channel becoming open in a given unit time; (6) increasing the time duration of the GABA receptor ion channel stays open in a given unit time; (7) increasing the dimension of an opened GABA receptor ion channel; or any combinations of mechanism (1)-(7).

In some embodiments, the GABA receptor is a GABA_{A}R. In some embodiments, the GABA_{A}R receptor comprises at least one GABA receptor subunit or a functional domain thereof. In some embodiments, the GABA_{A}R receptor comprises at least one of α(1-6), β(1-3), γ(1-3), δ, ε(1-3), θ, and π subunits, or a functional domain thereof. In some embodiments, the GABA_{A}R receptor comprises at least one of α (1, 2, 3 or 5), β and γ subunits, or a functional domain thereof. In some embodiments, the GABA_{A}R receptor comprises at least one GABA receptor subunit selected from α (4/6), β, δ subunits, or a functional domain thereof. In some embodiments, the GABA_{A}R receptor comprises at least one GABA receptor subunit selected from 2α, 2β and 1γ (or 1δ) GABA receptor subunits, or one or more functional domains thereof. In some embodiments, the GABA_{A}R receptor comprises at least α1, β2 and γ2 GABA receptor subunits, or one or more functional domains thereof. In some embodiments, the GABA_{A}R receptor comprises at least α4, β3 and δ GABA receptor subunits, or one or more functional domains thereof.

In another aspect, the invention provides a membrane progesterone receptor (mPR) agonist as described herein for use in treating a CNS-related condition or disorder in a subject as described herein. In yet another aspect, the invention provides a membrane progesterone receptor (mPR) agonist and a GABAergic modulator as described herein for use in treating a CNS-related condition or disorder in a subject as described herein.

In another aspect, the invention provides the use of a membrane progesterone receptor (mPR) agonist as described herein for the preparation of a medicament for treating a CNS-related condition or disorder in a subject as described herein. In yet another aspect, the invention provides the use of a membrane progesterone receptor (mPR) agonist and a GABAergic modulator as described herein for the preparation of a medicament for treating a CNS-related condition or disorder in a subject as described herein.

### CNS Disorders

According to the present disclosure, the methods, therapeutic agents and compositions of this invention are useful in treating a CNS-related condition or disorder in a subject, including but not limited to, a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, a female sex dysfunction and/or a neurodegenerative disease and/or disorder.

### Psychiatric Disorders

### Mood Disorders

Provided herein are methods, therapeutic agents and compositions for treating a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

Premenstrual dysphoric disorder (PMDD) refers to a severe, at times disabling extension of premenstrual syndrome (PMS). PMDD causes extreme mood shifts with symptoms that typically begin seven to ten days before a female's period starts and continues for the first few days of a female's period. Symptoms include sadness or hopelessness, anxiety or tension, extreme moodiness, and marked irritability or anger.

### Anxiety Disorders

Also provided herein are methods, therapeutic agents and compositions for treating an anxiety disorder. Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausca, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And, in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Eating Disorders

The compounds described herein can be used in a method described herein, for example in the treatment of a disorder described herein such as an eating disorder. Eating disorders feature disturbances in eating behavior and weight regulation, and are associated with a wide range of adverse psychological, physical, and social consequences. An individual with an eating disorder may start out just eating smaller or larger amounts of food, but at some point, their urge to cat less or more spirals out of control. Eating disorders may be characterized by severe distress or concern about body weight or shape, or extreme efforts to manage weight or food intake. Eating disorders include anorexia nervosa, bulimia nervosa, binge-eating disorder, cachexia, and their variants.

Individuals with anorexia nervosa typically see themselves as overweight, even when they are underweight. Individuals with anorexia nervosa can become obsessed with eating, food, and weight control. Individuals with anorexia nervosa typically weigh themselves repeatedly, portion food carefully, and eat very small quantities of only certain foods. Individuals with anorexia nervosa may engage in binge eating, followed by extreme dieting, excessive exercise, self-induced vomiting, or misuse of laxatives, diuretics, or enemas. Symptoms include extremely low body weight, severe food restriction, relentless pursuit of thinness and unwillingness to maintain a normal or healthy weight, intense fear of gaining weight, distorted body image and self-esteem that is heavily influenced by perceptions of body weight and shape, or a denial of the seriousness of low body weight, lack of menstruation among girls and women. Other symptoms include the thinning of the bones, brittle hair and nails, dry and yellowish skin, growth of fine hair all over the body, mild anemia, muscle wasting, and weakness, severe constipation, low blood pressure or slowed breathing and pulse, damage to the structure and function of the heart, brain damage, multi-organ failure, drop in internal body temperature, lethargy, sluggishness, and infertility.

Individuals with bulimia nervosa have recurrent and frequent episodes of eating unusually large amounts of food and feel a lack of control over these episodes. This binge eating is followed by behavior that compensates for the overeating such as forced vomiting, excessive use of laxatives or diuretics, fasting, excessive exercise, or a combination of these behaviors.

Unlike anorexia nervosa, people with bulimia nervosa usually maintain what is considered a healthy or normal weight, while some are slightly overweight. But like people with anorexia nervosa, they typically fear gaining weight, want desperately to lose weight, and are unhappy with their body size and shape. Usually, bulimic behavior is done secretly because it is often accompanied by feelings of disgust or shame. The binge eating and purging cycle can happen anywhere from several times a week to many times a day. Other symptoms include chronically inflamed and sore throat, swollen salivary glands in the neck and jaw area, worn tooth enamel, and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (that can lead to a heart attack or stroke).

Individuals with binge-eating disorder lose control over their eating. Unlike bulimia nervosa, periods of binge eating are not followed by compensatory behaviors like purging, excessive exercise, or fasting. Individuals with binge-eating disorder often are overweight or obese. Obese individuals with binge-eating disorder are at higher risk for developing cardiovascular disease and high blood pressure. They also experience guilt, shame, and distress about their binge eating, which can lead to more binge eating.

Cachexia is also known as “wasting disorder,” and is an eating-related issue experienced by many cancer patients. Individuals with cachexia may continue to cat normally, but their body may refuse to utilize the vitamins and nutrients that it is ingesting, or they will lose their appetite and stop eating. When an individual experiences loss of appetite and stops eating, they can be considered to have developed anorexia nervosa.

### Epilepsy

The therapeutic agents and compositions described herein can be used for example in the treatment of a disorder described herein such as epilepsy, status epilepticus, or seizure, for example as described in WO2013/112605 and WO/2014/031792, the contents of which are incorporated herein in their entirety.

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

The therapeutic agents and compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Psychotic Disorders

Also provided herein are methods, therapeutic agents and compositions for treating a psychotic disorder. The term “psychotic disorders” as used herein refers to a group of illnesses that affect the mind. These illnesses alter a patient's ability to think clearly, make good judgments, respond emotionally, communicate effectively, understand reality, and behave appropriately. When symptoms are severe, patient with psychotic disorders have difficulty staying in touch with reality and are often unable to meet the ordinary demands of daily life. Psychotic disorders include but are not limited to, schizophrenia, schizophreniform disorder, schizo-affective disorder, delusional disorder, brief psychotic disorder, shared psychotic disorder, psychotic disorder due to a general medical condition, substance-induced psychotic disorder or psychotic disorders not otherwise specified (Diagnostic and Statistical Manual of Mental Disorders, Ed. 4th, American Psychiatric Association, Washington, D.C. 1994).

### Impulse Control Disorders

Also provided herein are methods, therapeutic agents and compositions for treating an impulse control disorder. The term “impulse control disorders” as used herein refers to a class of psychiatric disorders characterized by impulsivity, i.e., failure to resist a temptation, an urge of an impulse. Five behavioral stages characterize impulsivity: an impulse, growing tension, pleasure on acting, relief from the urge and finally guilt (which may or may not arise).

Many psychiatric disorders feature impulsivity, including substance-related disorders, behavioral addictions, attention deficit hyperactivity disorder, antisocial personality disorder, borderline personality disorder, conduct disorder and some mood disorders. The fifth edition of the American Psychiatric Association's Diagnostic and statistical manual of mental disorders (DSM-5) that was published in 2013 includes a new chapter (not in DSM-IV-TR) on Disruptive, Impulse-Control, and Conduct Disorders covering disorders “characterized by problems in emotional and behavioral self-control.” It also includes Impulse-Control Disorders Not Elsewhere Classified, which encompasses intermittent explosive disorder, pyromania, and kleptomania.

### Neurological Disorders

### Neurodevelopmental Disorders

Also provided herein are methods, therapeutic agents and compositions for treating and/or preventing neurodevelopment disorders. Neurodevelopmental disorders are a group of disorders in which the development of the central nervous system is disturbed. This can include developmental brain dysfunction, which can manifest as neuropsychiatric problems or impaired motor function, learning, language or non-verbal communication.

Non-limiting examples of neurodevelopmental disorder include autism, autistic disorder, autistic spectrum disorder, Asperger syndrome, Pediatric Autoimmune Neuropsychiatric Disorders Associated with Streptococcal infections (PANDAS); Rett syndrome; Williams syndrome; Renpenning's syndrome; fragile X syndrome; Down syndrome; Prader-Willi syndrome; Sotos syndrome; Tuberous sclerosis complex (TSC); Timothy syndrome; Joubert syndrome; holoprosencephaly; Hirschsprung's disease; intestinal neuronal dysplasia; and Williams syndrome, pervasive developmental disorder, attention deficit hyperactivity disorder, deficits in attention, motor control and perception (DAMP), schizophrenia, obsessive-compulsive disorder, disorders affecting emotion, learning ability, and memory.

### Neurodegenerative Disorders

Also provided herein are methods, therapeutic agents and compositions for treating and/or preventing a neurodegenerative disease. The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating a neurodegenerative disease also include treating loss of neuronal function characteristic of neurodegenerative disorder.

### Neuroendocrine Disorders

Also provided herein are methods, therapeutic agents and compositions that can be used for treating neuroendocrine disorders and dysfunction. As used herein, “neuroendocrine disorder” or “neuroendocrine dysfunction” refers to a variety of conditions caused by imbalances in the body's hormone production directly related to the brain. Neuroendocrine disorders involve interactions between the nervous system and the endocrine system. Because the hypothalamus and the pituitary gland are two areas of the brain that regulate the production of hormones, damage to the hypothalamus or pituitary gland, e.g., by traumatic brain injury, may impact the production of hormones and other neuroendocrine functions of the brain. In some embodiments, the neuroendocrine disorder or dysfunction is associated with a women's health disorder or condition (e.g., a women's health disorder or condition described herein). In some embodiments, the neuroendocrine disorder or dysfunction is associated with a women's health disorder or condition is polycystic ovary syndrome.

Symptoms of neuroendocrine disorder include, but are not limited to, behavioral, emotional, and sleep-related symptoms, symptoms related to reproductive function, and somatic symptoms; including but not limited to fatigue, poor memory, anxiety, depression, weight gain or loss, emotional lability, lack of concentration, attention difficulties, loss of lipido, infertility, amenorrhea, loss of muscle mass, increased belly body fat, low blood pressure, reduced heart rate, hair loss, anemia, constipation, cold intolerance, and dry skin.

### Movement Disorders

Also provided herein are methods, therapeutic agents and compositions for treating a movement disorder. Movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea, tremor), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis), essential tremor, Stiff-Person syndrome, spasticity, Freidrich's ataxia, Cerebellar ataxia, dystonia, Tourette Syndrome, Fragile X-associated tremor or ataxia syndromes, drug-induced or medication-induced Parkinsonism (such as neuroleptic-induced acute akathisia, acute dystonia, Parkinsonism, or tardive dyskinesia, neuroleptic malignant syndrome, or medication-induced postural tremor), etc.

Tremor is an involuntary, at times rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occurs in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Ataxia includes cerebellar ataxia (McLeod neuroacanthocytosis syndrome (MLS), levodopa-induced dyskinesia.

### Sleep Disorders

Also provided herein are methods, therapeutic agents and compositions for treating a sleep disorder. The term “sleep disorder” is meant to refer to generally any abnormal sleeping pattern. Some sleep disorders are serious enough to interfere with normal physical, mental, social and emotional functioning. Sleep disorders are broadly classified into insomnia, dyssomnias, parasomnias, circadian rhythm sleep disorders involving the timing of sleep, and other disorders including ones caused by medical or psychological conditions and sleeping sickness. Non-limiting examples of sleep disorders include circadian rhythm abnormality, insomnia, parasomnia, sleep apnea syndrome, narcolepsy and hypersomnia, rapid eye movement behavior disorder, restless legs syndrome, periodic leg movements of sleep, obstructive sleep apnea, central sleep apnea, snoring, nightmares, sleep terrors, sleepwalking, confusional arousals, sleep paralysis, sleep eating disorder, or narcolepsy (See, for example, C G Goetz (editor), Textbook of Clinical Neurology, 3rd Edition, 2007, Chapter 54).

### Seizure

Also provided herein are methods, therapeutic agents and compositions for treating a seizure. A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

Seizure described herein can include focal seizures with either motor (automatisms, atonic, clonic, epileptic spasms, hyperkinetic, myoclonic, and tonic) or non-motor (autonomic, behavioral arrest, cognition, emotional, and sensory) onset, generalized seizures with either motor (tonic-clonic, clonic, myoclonic, myoclonic-tonic-clonic, myoclonic-atonic, atonic, epileptic spasms) or non-motor (absence) onset, seizures with unknown motor (tonic-clonic, epileptic spasms) or non-motor (behavioral arrest) onset; seizures associated with clinical syndromes, such as Dravet syndrome, Rett syndrome, Lennox Gasteau syndrome, Tuberous sclerosis, Angelmans syndrome, catamenial epilepsy.

### Neuroinflammatory Disorders

Also provided herein are methods, therapeutic agents and compositions for treating a neuroinflammatory disorder. The term “neuroinflammatory disorder” designates a disease having a neuroinflammation component such as, in particular a neurodegenerative, autoimmune, infectious, toxic or traumatic disorder, where inflammatory component could be aetiological or pathology-exacerbating factor. Said neurodegenerative, autoimmune, infectious, toxic or traumatic diseases with inflammatory component include multiple sclerosis (MS), Alzheimer's disease (AD), Parkinson's disease (PD), Amyotrophic lateral sclerosis (ALS), Acute disseminated encephalomyelitis (ADEM) and Neuromyelitis optica (NMO).

### Analgesia/Sedation

Also provided herein are methods, therapeutic agents and compositions for inducing anesthesia, analgesia and sedation. Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands. Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patent airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patent airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patent airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 week, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

### Sensory Deficit Disorders

Also provided herein are methods, therapeutic agents and compositions for treating a sensory deficient disorder. Sensory processing disorder is a condition in which the brain has trouble receiving and responding to information that comes in through the senses. Non-limiting examples of sensory deficit disorders include tinnitus, synesthesia, hearing impairment and loss. Pathological brain function in sensory-deficit disorders has been associated with nicotinic cholinergic transmission particularly through oc7 receptors (Freedman R et al., Biol. Psychiatry, 1995, 38, 22-33; Tsuang D W et al., Am. J. Med. Genct., 2001, 105, 662-668; Carson R et al., Neuromolecular, 2008, Med. 10, 377-384; Leonard S et al., Pharmacol. Biochem. Behav., 2001, 70, 561-570; Freedman R et al., Curr. Psychiatry Rep., 2003, 5, 155-161; Cannon T O et al., Curr. Opin. Psychiatry, 2005, 18, 135-140). A defective pre-attention processing of sensory information is understood to be the basis of cognitive fragmentation in schizophrenia and related neuropsychiatric disorders (Leiser S C et al., Pharmacol. Ther., 2009, 122, 302-31 1). Genetic linkage studies have traced sharing of the oc7 gene locus for several affective, attention, anxiety and psychotic disorders (Leonard S et al., Pharmacol. Biochem. Behav., 2001, 70, 561-570; Suemaru K et al., Nippon Yakurigaku Zasshi, 2002, 1 19, 295-300).

### Neuroprotection, Glaucoma, Metabolic Disorders

Also provided herein are methods, therapeutic agents and compositions for treating a neuroprotection disorder, a metabolic disorder, such as glaucoma. The term “glaucoma” refers to an ocular disease in which the optic nerve is damaged in a characteristic pattern. This can permanently damage vision in the affected eye and lead to blindness if left untreated. It is normally associated with increased fluid pressure in the eye (aqueous humor). The term ocular hypertension is used for patients with consistently raised intraocular pressure (TOP) without any associated optic nerve damage. Conversely, the term normal tension or low tension glaucoma is used for those with optic nerve damage and associated visual field loss but normal or low TOP. The nerve damage involves loss of retinal ganglion cells in a characteristic pattern. There are many different subtypes of glaucoma, but they can all be considered to be a type of optic neuropathy. Raised intraocular pressure (e.g., above 21 mmHg or 2.8 kPa) is the most important and only modifiable risk factor for glaucoma. However, some may have high eye pressure for years and never develop damage, while others can develop nerve damage at a relatively low pressure. Untreated glaucoma can lead to permanent damage of the optic nerve and resultant visual field loss, which over time can progress to blindness.

### Female Sexual Dysfunction

Also provided herein are methods, therapeutic agents and compositions for treating a female sexual disorder. “Female sexual dysfunctions” or “female sexual disorders” are defined in The Diagnostic and Statistical Manual of Mental Disorders (“DSM”), and include the following three categories: (1) Genitopelvic pain/penetration disorder; (2) Sexual interest/arousal disorder; and (3) Female orgasmic disorder. All of these afflictions are common and known to significantly reduce the quality of life for millions of women and their sexual partners. In the most current version of DSM, (DSM-V), older terminologies, both Hypoactive Sexual Desire Disorder (HSDD) and Female Arousal Disorder were merged into a single category of female sexual disorder now called Sexual Interest/Arousal Disorder (SIAD). Similarly, the formerly separate dyspareunia and vaginismus disorders are now collectively called Genitopelvic Pain/Penetration Disorder (GPPD). The terminology Female Orgasmic Disorder (FOD) remains unchanged.

The DSM-V defines genitopelvic pain/penetration disorder, (GPPD) as difficulty in vaginal penetration, marked vulvovaginal or pelvic pain during penetration, or attempt at penetration (dyspareunia), fear or anxiety about pain in anticipation of, during, or after penetration, and tightening or tensing of pelvic floor muscles during attempted penetration (vaginismus). For many women, however, pain may occur outside the context of penetration or sexual intercourse. For example, pain or discomfort may occur during any manipulation of their external genitalia (a condition known as vulvodynia). Such pain or discomfort may be due to Vulvovaginal atrophy (VVA), including thinning of the vulvovaginal epithelium and a lack of lubrication and/or may be characterized as vulvodynia.

This condition may manifest itself as primary vestibulodynia (i.e. pain with first attempted tampon and/or intercourse) or secondary vestibulodynia (i.e. development of vulvar pain following previously painless tampon use and/or intercourse).

Another contributing cause of genital pain/penetration disorder (GPPD) is known as vestibulodynia, vulvodynia and/or vestibulitis. This affliction may be seen in up to 15% of all premenopausal women sometime in their lifetime. It is also a very common affliction of postmenopausal women unrelated to VVA.

Female vestibulodynia may be generally described as a disorder of unknown etiology where there is localized provoked vulvar pain upon penetration of the vagina. There is also tenderness to touch around the vaginal opening (vestibule) during normal self or partner's manual sexual contact or during a health professional's physical examination. The entire area around the vaginal introitus (vulvodynia) can be affected but the experienced discomfort or pain is most commonly pronounced in the localized area of the posterior forchette (vestibulodynia). The affected tissue in the vestibule has increased nerve endings and signs of inflammation and is typically painful. It occurs in women of all ages.

The two other disorders of female sexual dysfunction are: sexual interest/arousal disorder (SIAD) and female orgasmic disorder (FOD). Sexual Interest/Arousal Disorder (“SIAD”) as specified in the DSM refers to “the persistent or recurrent inability to attain or to maintain sufficient sexual excitement, which causes personal distress.” In addition to absent or decreased sexual interest, including erotic thoughts or fantasies, there are four criteria that are taken into account to determine whether a woman suffers from SIAD. A woman has SIAD if she experiences personal distress caused by a decrease or lack of at least three of the following four criteria: 1) initiation of sexual activity or responsiveness to a partner's attempts to initiate it, 2) excitement and pleasure, 3) response to sexual cues, and 4) sensations during sexual activity, whether genital or non-genital. Again, three of the foregoing criteria are required for diagnosis. It may be expressed generally as lack of subjective excitement or lack of genital (lubrication/swelling) or other somatic responses.

Female orgasmic disorder, (FOD) as defined in the DSM, is the absence (anorgasmia), infrequency or delay of orgasm, and/or reduced intensity of said orgasm. Such orgasmic dysfunction may also occur when a woman has difficulty reaching orgasm, even when sexually aroused with sufficient sexual stimulation. Many women have difficulty reaching orgasm with a partner, or during masturbation, even after ample sexual stimulation. Female Orgasmic Disorder (FOD) affects approximately one in three women.

It can be difficult to determine the particular underlying cause of Female Orgasmic Disorder (FOD). Women may have difficulty reaching orgasm due to one or more physical, emotional, and/or psychological factors. Contributing factors include: older age, medical conditions, such as diabetes, a history of gynecological surgeries, such as a hysterectomy, the use of certain medications, particularly selective serotonin reuptake inhibitors (SSRIs), mental health conditions, such as depression or anxiety, stress, societal negative stereotypes of women's sexuality, lack of adequate or effective sexual stimulation, etc. Sometimes, a combination of these factors can make achieving an orgasm difficult or not possible.

The inability to orgasm can lead to distress, which may make it even more difficult to achieve orgasm in the future. The main symptom of orgasmic disorder is the inability to achieve sexual climax. Women with female orgasmic disorder (FOD) may have difficulty achieving orgasm during either sexual intercourse or during masturbation. For many women, having unsatisfying orgasms, less intense orgasms, or taking longer than desirable to reach climax are common symptoms of FOD that lead to emotional distress.

### Pharmaceutical Composition

In another aspect, provided herein are pharmaceutical compositions comprising a therapeutic agent of the present invention (also referred to as the “active ingredient”) and a pharmaceutically acceptable excipient. In some embodiments, the pharmaceutical composition comprises an effective amount of the active ingredient. In some embodiments, the pharmaceutical composition comprises a therapeutically effective amount of the active ingredient. In some embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. In some embodiments, the effective amount is sufficient to beneficially alter the CNS-related condition or disorder in said subject. The amount of the therapeutic agent actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the therapeutic agent in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The therapeutic agents provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The therapeutic agents of the present invention can also be administered in sustained release forms or from sustained release drug delivery systems. A description of representative sustained release materials can be found in Remington's Pharmaceutical Sciences.

The present invention also relates to the pharmaceutically acceptable formulations of a compound of the present invention. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether (β-cyclodextrin, e.g., for example, sulfobutyl ether (β-cyclodextrin, also known as Captisol®. See, e.g., U.S. Pat. No. 5,376,645. In certain embodiments, the formulation comprises hexapropyl-β-cyclodextrin (e.g., 10-50% in water).

### EXAMPLES

### Example 1 Recombinant GABA_{A} Pharmacology

Cellular electrophysiology was used to measure the pharmacological properties of ALLO, ganaxolone and SGE-516 in heterologous cell systems. Compounds were tested for their ability to affect GABA mediated currents at a submaximal agonist dose (GABA EC₂₀=2 μM).

LTK cells were stably transfected with the α1β2γ2 subunits of the GABA receptor and CHO cells are transiently transfected with the α4β3γ subunits via the Lipofecatamine method. Cells were passaged at a confluence of about 50-80% and then seeded onto 35 mm sterile culture dishes containing 2 ml culture complete medium without antibiotics or antimycotics. Cells were cultivated at a density that enabled the recording of single cells without visible connections to other cells.

Whole-cell currents were measured with HEKA EPC-10 amplifiers using PatchMaster software. Bath solution for all experiments contained (in mM): NaCl 137, KCl 4, CaCl₂ 1.8, MgCl₂ 1, HEPES 10, D-Glucose 10, pH 7.4 with NaOH. Intracellular (pipette) solution contained (in mM): KCl 130, MgCl2 1, Mg-ATP 5, HEPES 10, EGTA 5, pH 7.2 with KOH. During experiments, cells and solutions were maintained at room temperature (19° C.-30° C.). For manual patchclamp recordings, cell culture dishes were placed on the dish holder of the microscope and continuously perfused (1 ml/min) with bath solution. After formation of a Gigaohm seal between the patch electrode and the cell (pipette resistance range: 2.5 MΩ-6.0MΩ; seal resistance range: >1 GΩ) the cell membrane across the pipette tip was ruptured to assure electrical access to the cell interior (whole-cell patch configuration).

Cells were voltage-clamped at a holding potential of −80 mV. GABA_{A} receptors were activated by 2 μM GABA and compounds were sequentially applied at increasing concentrations for 30 s prior to a 2 s application of GABA. GABA and compounds were applied to cells via the Dynaflow perfusion system (Cellectricon, Sweden). Test compounds were dissolved in DMSO to form 10 mM stock solution and serially diluted to 0.01, 0.1, 1, and 10 μM in bath solution. There was no effect on GABA currents when DMSO was applied to cells at its maximal concentration in solution (0.1%). All concentrations of test compound were tested on each cell. The relative percentage potentiation was defined as the peak amplitude in response to GABA EC20 in the presence of test compound divided by the peak amplitude in response to GABA EC20 alone, multiplied by 100.

### Hippocampal Slice Preparation

Brain slices were prepared from 3- to 5-week-old male C57 mice. Mice were anesthetized with isoflurane, decapitated, and brains were rapidly removed and submerged in ice-cold cutting solution containing (mM): 126 NaCl, 2.5 KCl, 0.5 CaCl₂, 2 MgCl₂, 26 NaHCO₃, 1.25 NaH₂PO₄, 10 glucose, 1.5 sodium pyruvate, and 3 kynurenic acid. Coronal 310 μm thick slices were cut with the vibratome VT1000S (Leica Microsystems, St Louis, MO, USA). The slices were then transferred into incubation chamber filled with prewarmed (31-32° C.) oxygenated artificial cerebro-spinal fluid (ACSF) of the following composition (in mM): 126 NaCl, 2.5 KCl, 2 CaCl₂), 2 MgCl₂, 26 NaHCO₃, 1.25 NaH₂PO₄, 10 glucose, 1.5 sodium pyruvate, 1 glutamine, 3 kynurenic acid and 0.005 GABA bubbled with 95% O₂-5% CO₂. Slices were allowed to recover at 32° C. for at least 1 hr before recording. Exogenous GABA was added in an attempt to standardize ambient GABA in the slice and provide an agonist source for newly inserted extrasynaptic GABA_{A}Rs.

### Example 2 Electrophysiology Recordings

After recovery, a single slice was transferred to a submerged, dual perfusion recording chamber (Warner Instruments, Hamden, CT, USA) on the stage of an upright microscope (Nikon FN-1) with a 40× water immersion objective equipped with DIC/IR optics. Slices were maintained at 32° C. and gravity-superfused with ACSF solution throughout experimentation and perfused at rate of 2 ml/min with oxygenated (O₂/CO₂ 95/5%) ACSF.

Whole-cell currents were recorded from the dentate gyrus granule cells (DGGCs) in 310-μm-thick coronal hippocampal slices. Patch pipettes (5-7M (2) were pulled from borosilicate glass (World Precision Instruments) and filled with intracellular solution of the composition (in mM) as follows: 140 CsCl, 1 MgCl2, 0.1 EGTA, 10 HEPES, 2 Mg-ATP, 4 NaCl and 0.3 Na-GTP (pH=7.2 with CsOH). A 5 min period for stabilization after obtaining the whole-cell recording conformation (holding potential of −60 mV) was allowed before currents were recorded using an Axopatch 200B amplifier (Molecular Devices), low-pass filtered at 2 kHz, digitized at 20 kHz (Digidata 1440A; Molecular Devices), and stored for offline analysis.

### Example 3 Electrophysiology Analysis

For tonic current measurements, an all-points histogram was plotted for a 10 s period before and during 100 μM picrotoxin application, once the response reached a plateau level. Recordings with unstable baselines were discarded. Fitting the histogram with a Gaussian distribution gave the mean baseline current amplitude and the difference between the amplitudes before and during picrotoxin was considered to be the tonic current. The negative section of the all-points histogram which corresponds to the inward IPSCs was not fitted with a Gaussian distribution (Kretschmannova et al., 2013; Nusser and Mody, 2002). Series resistance and whole-cell capacitance were continually monitored and compensated throughout the course of the experiment. Recordings were eliminated from data analysis if series resistance increased by >20%. Spontaneous inhibitory post-synaptic currents (sIPSCs) were analyzed using the mini-analysis software (version 5.6.4; Synaptosoft, Decatur, GA). Minimum threshold detection was set to 3 times the value of baseline noise signal. To assess sIPSC kinetics, the recording trace was visually inspected and only events with a stable baseline, sharp rising phase, and single peak were used to negate artifacts due to event summation. Only recordings with a minimum of 200 events fitting these criteria were analyzed. sIPSCs amplitude, and frequency from each experimental condition was pooled and expressed as mean 1 SEM. To measure sIPSC decay, 100 consecutive events were averaged and the decay was fitted to a double exponential and the weighted decay constant (tw) was determined. Statistical analysis was performed by using Student t-test (paired and unpaired where appropriate), where p<0.05 is considered significant.

### Example 4 Metabolic Labeling and Biotinylation

Hippocampi were dissected out of acute slices from 8 to 12 week old C57/β16 mice and lysed with phosphate buffer including: 20 mM Tris-HCl, 150 mM NaCl, 5 mM EDTA, 10 mM NaF, 2 mM Na3 VO4, 10 mM pyrophosphate, 0.1% SDS and 1% Triton after drug treatment. The β3 subunit was isolated using immunoprecipitation with β3 antibodies, after correction for protein content and the specific activity of labeling. Results were attained by SDS/PAGE followed by autoradiography (Abramian et al., 2010). For biotinylation experiments hippocampi were dissected out of acute slices from 8 to 12 week old C57/β16 mice and incubated in artificial cerebrospinal fluid (ACSF) described above at 30° C. for 1 h for recovery before experimentation. Slices were then placed on ice and incubated for 30 min with 1 mg/mL NHS-SS-biotin (Pierce). Excess biotin was removed by a 50 mM glycine quenching buffer, followed by washing of slices three times in ice-cold ACSF. The tissue was snap frozen on dry ice for 5 min, thawed at 4° C. and lysed. The lysates were solubilized with 2% Triton at 4° C. on a rotating wheel for 1 h. The insoluble material was removed by centrifugation, and 350-500 μg of protein lysate were incubated with NeutrAvidin beads (Pierce) for 18-24 h at 4° C. Bound material was eluted with sample buffer and subjected to SDS/PAGE and then immunoblotted with the indicated antibodies. Blots were then quantified using the CCD-based ChemiDoc XRS b system. Antibodies against the β3 subunit and the phospho-β3 antibody were generated and verified by the laboratory of S. M. (Vien et al., 2015).

### Example 5: Comparing the Effects of Endogenous and Synthetic Neurosteroids on the Activity of Recombinant GABA_{A}Rs

ALLO and ganaxolone are known PAMs of both synaptic and extrasynaptic GABA_{A}R-mediated currents. The ability of candidate compound SGE-516 to act as a PAM was compared to ALLO and ganaxolone using the whole-cell recordings of recombinant human GABA_{A} receptors expressed in mammalian cells. The α1β2γ2 or α4β3δ subunit combinations were chosen as representatives of typical synaptic and extrasynaptic GABAA receptors respectively. Similar to previous reports (Botella et al., 2015), ALLO, ganaxolone and SGE-516 allosterically potentiated currents induced by EC₂₀ concentration of GABA in a concentration-dependent manner in both synaptic- and extrasynaptic-type GABA_{A}Rs. ALLO potentiated α1β2γ2 receptors with an EC₅₀ of 115 nM and Eₘₐₓ of 229% and potentiated α4β3δ receptors with an EC₅₀ of 57 nM and Eₘₐₓ of 426%. SGE-516 potentiated α1β2γ2 receptors with an EC₅₀ of 61 nM and Emax of 219%. Likewise, SGE-516 potentiated α4β3δ receptors with an EC₅₀ of 193 nM and Emax of 400%. Ganaxolone potentiated α1β2γ2 receptors with an EC₅₀ of 256 nM and Eₘₐₓ of 307% and potentiated α4β3δ receptors with an EC₅₀ of 94 nM and Eₘₐₓ of 225% (Table 1).

To confirm the presence of the γ2 subunit in α1β2γ2 receptors, the effects of diazepam (a benzodiazepine PAM specifically for synaptic GABA_{A}Rs) was evaluated (data not shown). It was observed that diazepam potentiates the GABA evoked currents in α1β2γ2 receptors with an EC₅₀ of 69 nM and Eₘₐₓ of 184%. In contrast, it did not alter GABA evoked currents in α4β3δ receptors. The results indicate that like ALLO, and ganaxolone, SGE-516 is a potent and efficacious PAM for both synaptic and extrasynaptic type GABA_{A}Rs.

TABLE 1
Properties of neuroactive steroids on recombinant GABA_{A}Rs.

| | α1β2γ2 | | α4β3δ | |
|---|---|---|---|---|
| | EC₅₀ | Eₘₐₓ ± | EC₅₀ | Eₘₐₓ ± |
| | (pEC₅₀ ± | S.E.M. | (pEC₅₀ ± | S.E.M. |
| Compound | S.E.M.) | (%) | S.E.M.) | (%) |
| ALLO | 115 (6.9 ± 0.2) | 229 ± 19 | 57 (7.2 ± 0.3) | 426 ± 42 |
| SGE-516 | 61 (7.2 ± 0.3) | 219 ± 21 | 193 (6.7 ± 0.1) | 505 ± 19 |
| Ganaxolone | 256 (6.6 ± 0.2) | 400 ± 27 | 94 (7.0 ± 0.1) | 225 ± 8 |

EC₅₀ values are given in nM with-pEC₅₀ ± S.E.M., n = 3 for all

### Example 6: Acute Exposure to NASs Allosterically Potentiates Tonic Current in the Dentate Gyrus Granule Cells

The results from the above experiments with synaptic-, and extrasynaptic-like GABA_{A}Rs expressed in HEK293 cells demonstrated the ability of ALLO, SGE-516, and ganaxolone to potentiate sub-maximal GABA-mediated currents. The ability of acute application of these NASs to allosterically modulate phasic and tonic currents in dentate gyrus granule cells (DGGCs) in hippocampal slices from 3 to 5 weeks old male C57/β16 mice was examined. Hippocampal slices from p21-35 (C57/β16) mice were allowed to recover for at least 1 h following slicing. Slices were transferred to the recording chamber of the icroscope. After achieving the whole-cell configuration approximately 10 min was allowed for membrane currents to stabilize. Hippocampal slices were acutely exposed to 100 nM ALLO, SGE-516, or ganaxolone (Ganax) for 10 min followed by 100 μM picrotoxin (PTX).

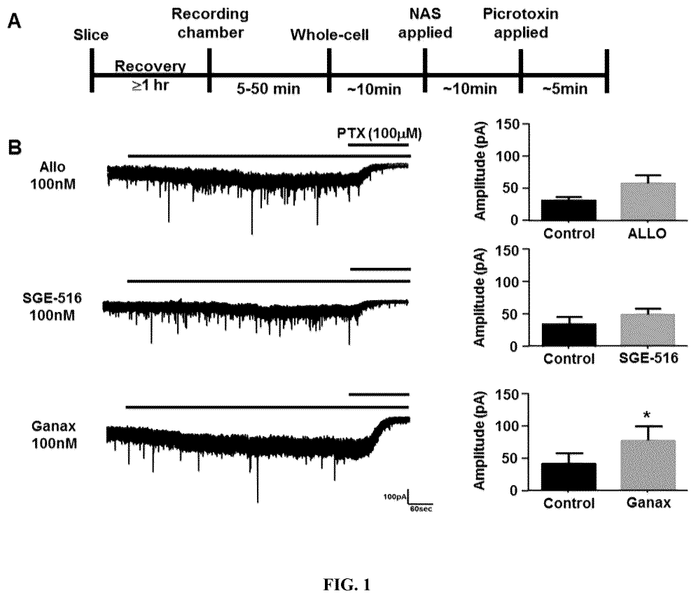

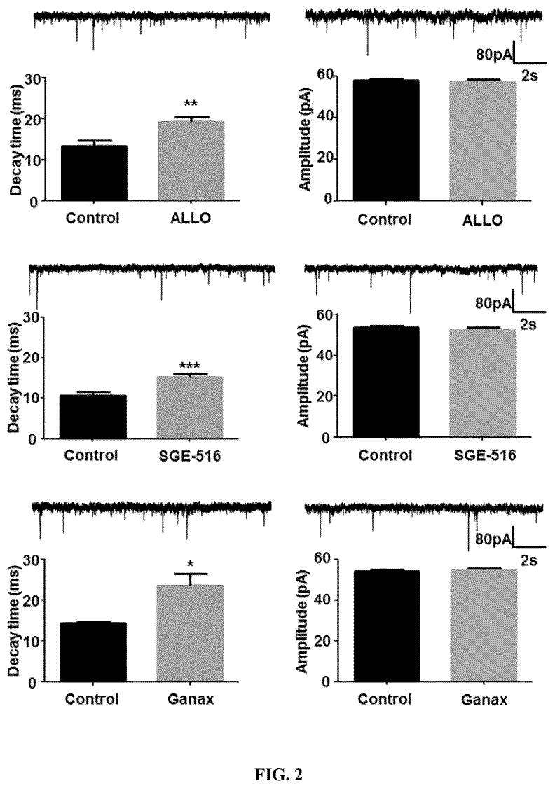

Both ALLO, and SGE-516 modulated the tonic holding current in DGGCs in hippocampal slices (FIG. 1). At 100 nM, ALLO modulated the tonic current from 31.8±5.9 pA, to 58.3±13.0 pA, (n=5) and 100 nM SGE-516 modulated the tonic current from 34.2±11.1 pA, to 49.2±8.9 (n=7). The only significant modulation was observed with 100 nM ganaxolone which modulated DGGC tonic current from 42.1±17.5 to 78.8±23.2 pA (n=6; p=0.004 paired t-test).

### Example 7: Comparing the Acute Effects of NASs on Phasic Currents in DGGCs

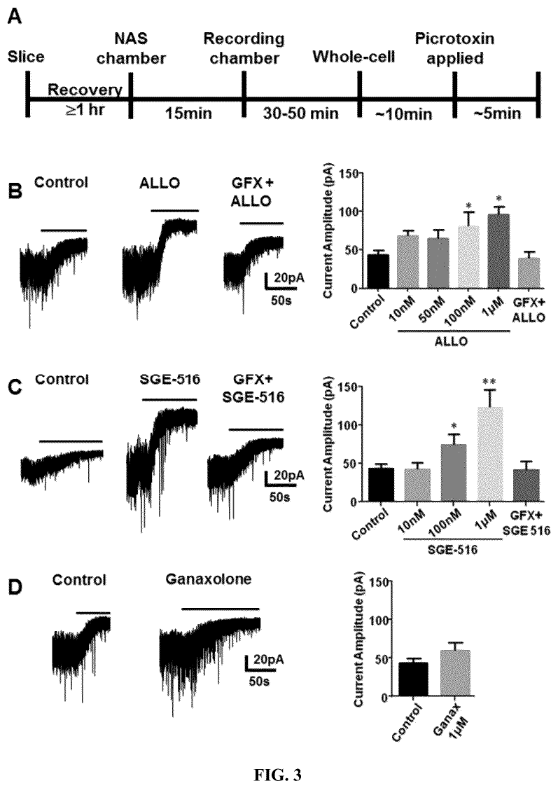

The properties of inhibitory synaptic currents in DGGCs before and during exposure to NASs (FIG. 2) were compared. It was observed that there was no significant difference in the mean sIPSC amplitude before and during exposure with 100 nM ALLO (p=0.85, n=5), 100 nM SGE-516 (p=0.46, n=7) and 100 nM ganaxolone (p=0.07, n=6) (Table 2). However, the mean sIPSC decay time significantly increased in the presence of ALLO, SGE-516, and ganaxolone (p=0.03, p=0.01, and p=0.04 respectively, Table 2).

TABLE 2
Allosteric modulation of DGGCs [symbol] evoked by 100 nM ALLO, SGE-516, and ganaxolone

| dPSC | Content | ALLO | | Control | SGE-516 | | Control | [symbol] | |
|---|---|---|---|---|---|---|---|---|---|
| Amplitude (pA) | 59.2 ± 0.7 | 39.1 | ± 0.6 | 53.7 ± 0.7 | 52.9 | ± 0.7 | 54.2 ± 0.6 | 55.1 | ± 0.7 |
| Frequency (Hz) | 4.5 ± 0.9 | 3.4 | ± 1.0 | 4.1 ± 0.4 | 4.6 | ± 0.6 | 6.4 ± 1.3 | 5.6 | ± 1.7 |
| Decay (ms) | 13.4 ± 1.3 | 19.2 | ± 1.3 [symbol] | 10.5 ± 1.1 | 15.1 | ± 0.8 [symbol] | 14.4 ± 0.5 | 23.8 | ± 2.9 [symbol] |

Data is mean SEM. [symbol] [symbol]
p = 0.01. [symbol]
p = 0.03. [symbol]
p = 0.04. [symbol]
indicates data missing or illegible when filed

### Example 8: Exposure to NASs Metabotropically Enhance Tonic Current in DGGCs

In addition to the allosteric modulation of GABA_{A} receptors, THDOC, exerts sustained effects on GABAergic tonic current by enhancing the PKC-dependent phosphorylation of the α4 and β3 subunits, leading to enhanced insertion and stability of GABA_{A}Rs into the membrane and a long lasting increase in tonic current (Abramian et al., 2010, 2014).

The sustained effects of ALLO, or the new synthetic NAS SGE-516 on the tonic current in DGGCs in hippocampal slices from 3 to 5 week old C57 male mice was analyzed. Hippocampal slices were allowed to recover for at least 1 h following slicing. Slices were then incubated for 15 min in a chamber containing NASs dissolved in ACSF. Slices were then transferred to the recording chamber of the microscope followed by a wash period between 30 and 60 min of continuous perfusion of NAS-free ACSF before recordings were started. Recordings were made from DGGCS in hippocampal slices from p21-35 C57 mice in the presence of 5 μM GABA followed by 100 μM picrotoxin and the difference in holding current was then determined. (FIG. 3A).

Slices exposed to ALLO, or SGE-516 demonstrated a concentration-dependent increase in the tonic current measured by the addition of picrotoxin with the maximal effects at 1 μM. Control, vehicle-treated slices had a tonic current of 43.9±5.7 pA (n=12), whereas the tonic currents for slices treated with SGE-516 (1 μM) was 123.0±22.2 pA, (n=6, p=0.0003), or ALLO (1 μM) was 95.8±10.8 pA (n=4, p=0.0005, FIG. 3B&C).

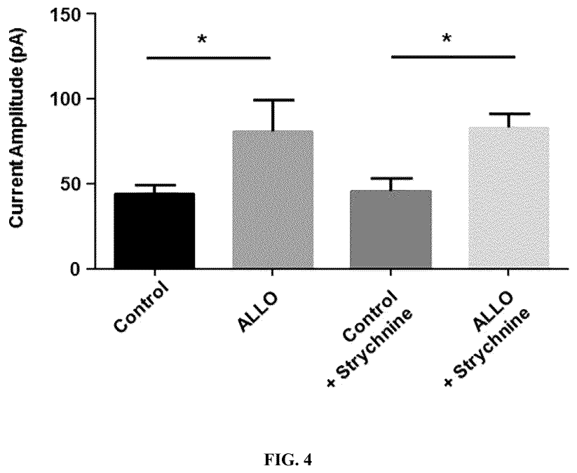

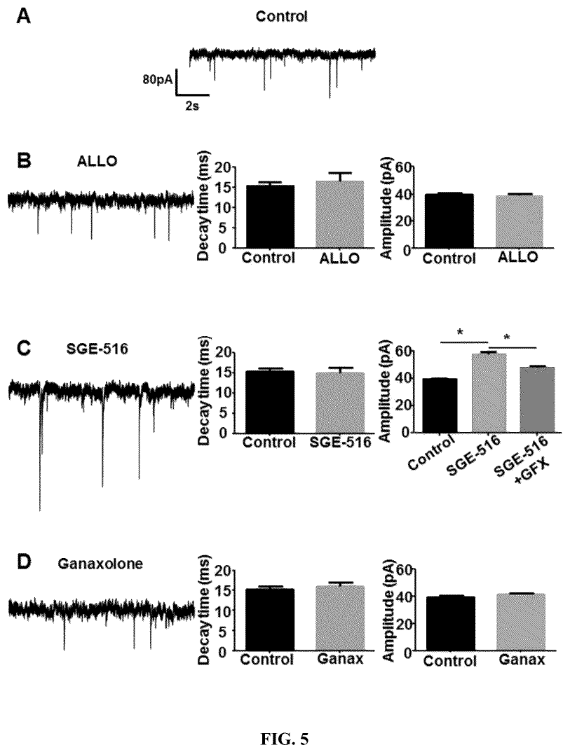

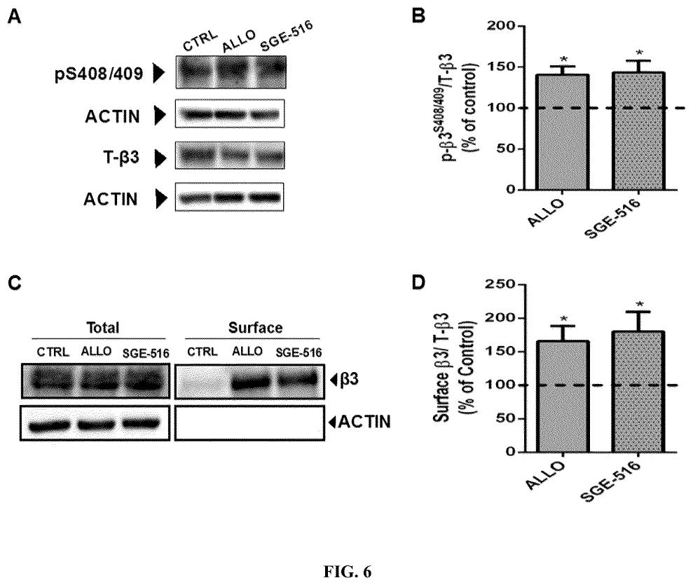

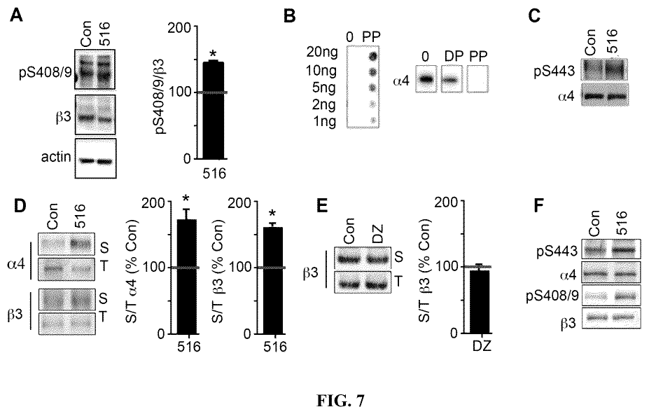

In addition, the metabotropic effect following 15 min incubation with synthetic NAS, ganaxolone was also examined. In contrast to the naturally occurring NASs THDOC, and ALLO, and the synthetic NAS, SGE-516, ganaxolone (1 μM) did not significantly alter the magnitude of tonic current in DGGCs (57.4±6.3 pA, n=7, p=0.14, FIG. 3D). To assess if the effects of NASs are dependent upon PKC, hippocampal slices were treated with the established PKC inhibitor GF 109203X (GFX 50 μM) for 15 min followed by co-exposure of to ALLO, or SGE-516 and GFX for 15 min. When tonic current was measured following ≥30 min washout, there was no significant difference to the tonic current measured in ALLO/GFX, or SGE-516/GFX treated slices with vehicle treated slices (FIG. 3). The tonic current was measured by blocking extrasynaptic GABA_{A}Rs with picrotoxin. Because picrotoxin also inhibits glycine receptors, the contribution of glycine receptors to tonic current was examined by using the specific glycine receptor inhibitor, strychnine. There was no difference in tonic current measured under control conditions in the absence or presence of strychnine (100 nM). Similarly, there was no difference in the increase in tonic current following a 15 min exposure to 100 nM ALLO in the absence or presence of strychnine (FIG. 4). These results suggest that glycine receptors have an undetected contribution to tonic current in DGGCs and that the metabotropic increase in tonic current by NAS exposure do not involve glycine receptors.

Collectively, these results suggest that the exposure of hippocampal slices to SGE-516 and ALLO has a strong sustained metabotropic effects on tonic current. In contrast, ganaxolone produce major effects via allosteric mechanism as compared to metabotropic effects.

### Example 9 Neurosteroids Increase Phosphorylation of GABA_{A}Rs and their Cell Surface Stability

Treatment of hippocampal slices with SGE-516 increased the phosphorylation of S408/9 measured using pS408/9 antibodies. To produce antibodies specific for phosphorylated S443, rabbits were injected with a synthetic peptide corresponding to residues 336-447 of the murine α4 subunit in which the serine residue corresponding to S443 was chemically phosphorylated; PGSLGSASTRPA. Hippocampal slices were treated with vehicle (Con), or SGE-516 for 20 min. Slices were immunoblotted with pS408/9, β3, or actin antibodies. The ratio of pS408/9/β3 immunoreactivity was then normalized to control slices (100%=the line). The resulting antiserum exhibited high titer against the immunogen (FIG. 7A) and was accordingly subjected to affinity purification.

Affinity purified pS443 was used to immunoblot varying concentrations of the immunizing phosphor-peptide (PP). pS443 was used to immunoblot extracts of hippocampal slices treated without preadsorption (0), preadsorbed with the dephosphorylated (DP), or phosphorylated antigen (PP). Immunoblotting hippocampal extracts with pS443 revealed the presence of a major band of 64 kDa, identical in migration to the α4 subunit. Moreover, the detection of this band was blocked by the phospho- but not the dephospho-antigen (FIG. 7B).

Hippocampal slices were treated with vehicle (Con) or 100 SGE-516 for 5 min and then immunoblotted with pS443 and α4 antibodies as indicated. pS443 immunoreactivity in hippocampal slices was increased by exposure to SGE-516, while total α4 levels were unaffected (FIG. 7C).

Hippocampal slices were treated as outlined above. Treated slices were then subject to biotinylation and lysis, and surface fractions were isolated on immobilized avidin. Surface(S) and total (T) fractions were immunoblotted with α4 and β3 subunit antibodies. The results showed that SGE-516 increased the plasma membrane accumulation of both the α4 and β3 subunits (FIG. 7D).

Slices were treated with 100 nM diazepam (DZ) and its effect on cell surface stability of the β3 subunit was determined as outlined above. Diazepam (DZ) did not affect cell surface stability of the β3 subunit (FIG. 7E).

IP injection of SGE-516 also increased phosphorylation of S443 and S408/9 in the brains of mice sacrificed by focused microwave irradiation (FIG. 7F). C57/β16 mice injected with SGE-516 (5 mg/kg IP), or vehicle. 30 min after the treatment mice were sacrificed by microwave irradiation. SDS-soluble hippocampal extracts were then immunoblotted with pS443, α4, pS408/9, or β3 subunit antibodies.

### Example 10: Mutation of S408/9 in the β3 Blocks the Ability of SGE-516 to Induce Sustained Effects on GABAergic Inhibition

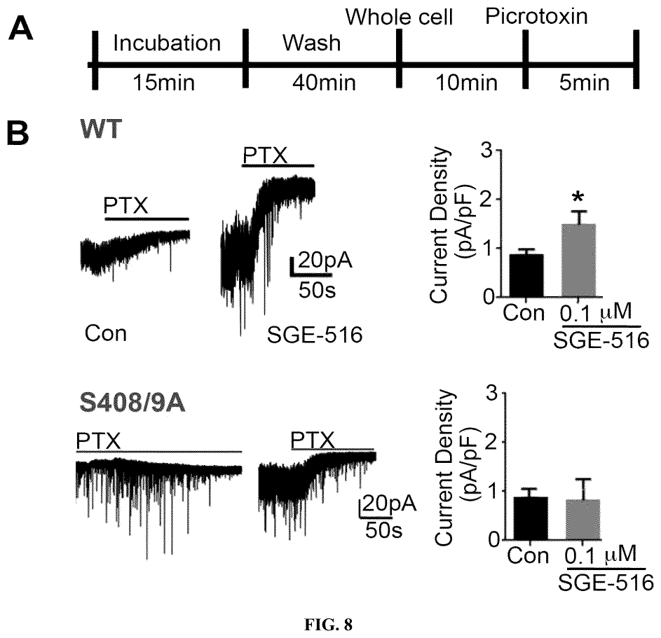

Consistent with the examples above, incubation of WT slices with 100 nM SGE-516 was followed by an extensive washout period. This treatment significant increased tonic current in DGGCs (FIG. 8A). In contrast, incubation of slices from S408/9A mice with SGE-516 under the same conditions did not modify tonic current in S408/9A mice (FIG. 8B).

### Example 11: Mutation of S408/9 in the β3 Blocks the Effects of SGE-516 on the Cell Surface levels og GABA_{A}Rs

Consistent with the examples above, hippocampal slices from WT and S408/9A mice were treated for 20 min with 100 nM SGE-516 or vehicle (Con) and subjected to biotinylation followed by immunoblotting with β3 and α4 subunit antibodies. The ratio of surface/total (S/T) immunoreactivity was then normalized to levels seen in control (100%).

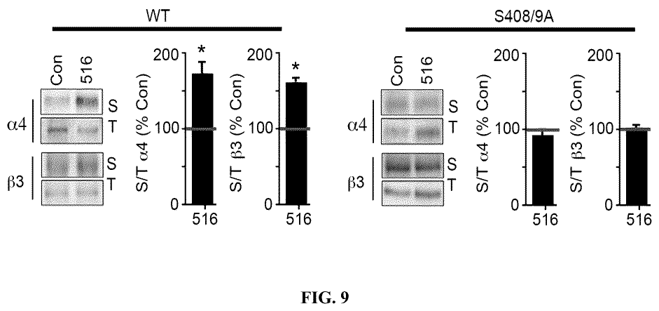

SGE-516 significantly increased the plasma membrane levels of GABA_{A}Rs containing the α4 and β3 subunits to 175-185% in hippocampal slices from WT mice (FIG. 9A). However, this effect was not seen in slices prepared from S408/9A mice (FIG. 9B).

### Example 12: Mutation of S408/9 in the β3 Subunit does not Block the Ability on Neurosteroids to Allosterically Modulate mIPSCs

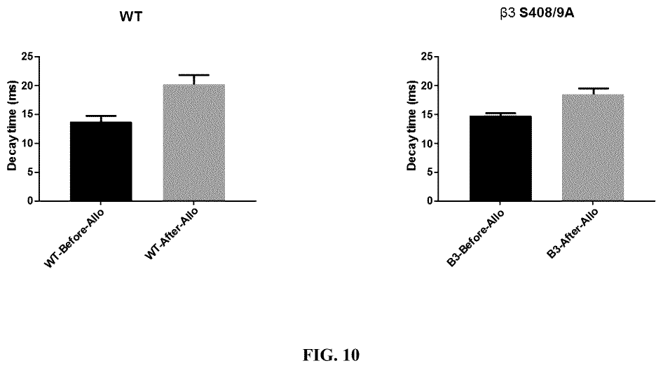

The ability of 100 nM ALLO to modulate the decay time of sIPSCs was compared in DGGCs from WT and S940A mice as detailed in FIG. 2. In contrast to WT, ALLO did not increase decay time in the mutant mice (FIG. 10).

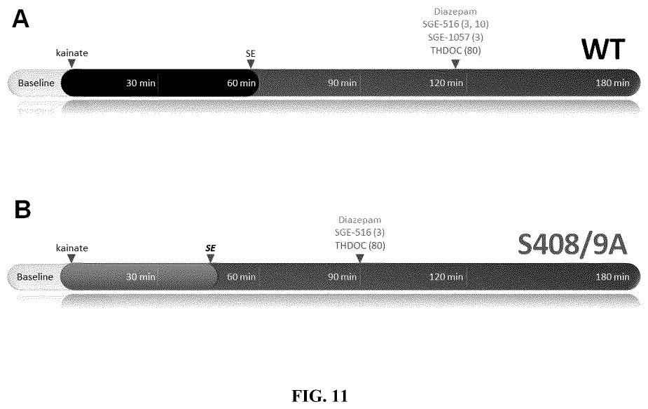

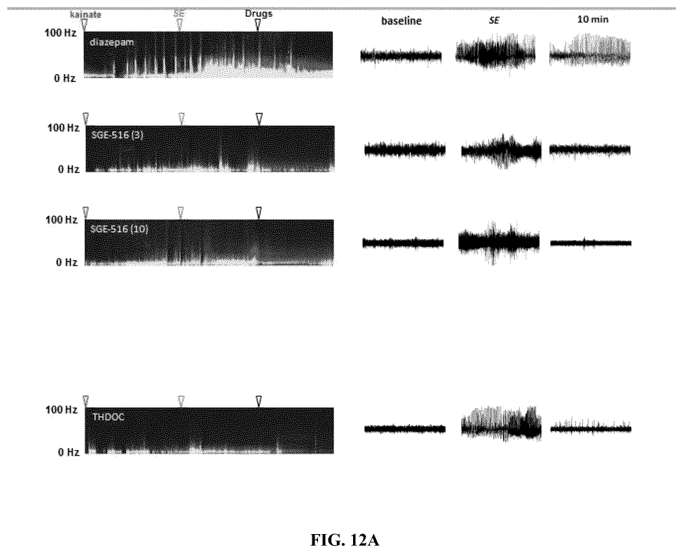

FIG. 11 shows the diagrams representing the protocols used to induced pharmacoresistant seizures in WT and S408/9A mice using kainite acid as measured using EEG recording. The time points used to test the anticonvulsant efficacy of benzodiazepines and neuroactive steroids to terminate seizure activity are also shown.

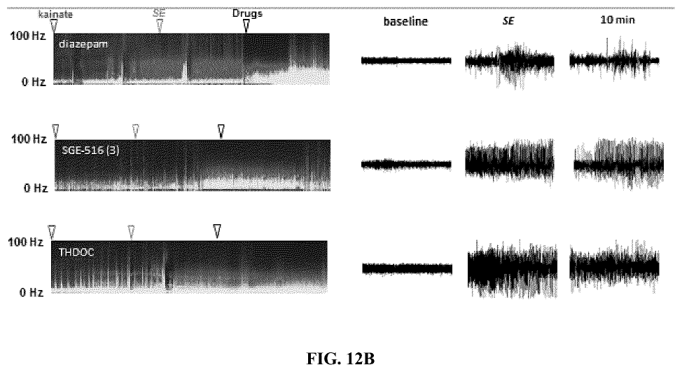

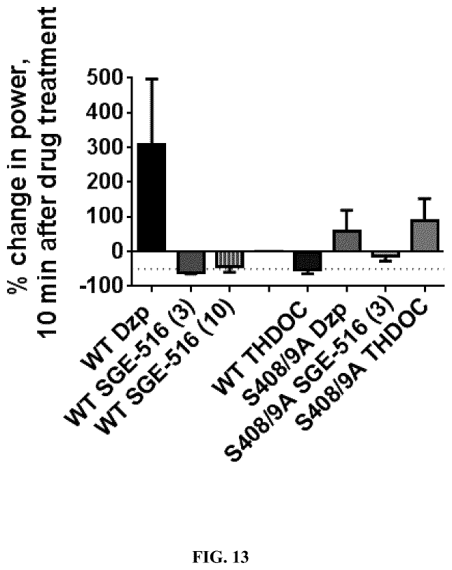

EEG power spectra are shown from WT mice undergoing SE induced by kainite >60 min (“SE” arrow), and FF. The ability of diazepam (10 mg/kg) SGE-516 (3 and 10 mg/kg) or THDOC (50 mg/kg). Representative EEG traces are shown at baseline, 60 after entrance into SE and 10 min after drug exposure. All drugs were injected IP as indicated by the “drug” arrow (FIG. 12A). The ability of diazepam, SGE-516 and THDOC to modify seizure activity in S408/9A mice was determined as detailed above (FIG. 12B). Seizure power was compared 10 min after exposure to the respective drugs. The only treatments that exhibited ≥50% reduction in power 10 minutes after treatment are SGE-517 (3 mg/kg) and THDOC (80 kg/mg) in wild type mice (FIG. 13).

### Example 13: Diversity in Ability of Neuroactive Steroids GABA PAM to Traffic GABA_{A} Receptors

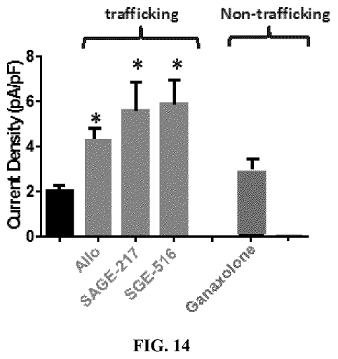

As shown in above examples, not all neurosteroids can modulate GABA receptor trafficking. As shown in FIG. 14, measured by the GABAergic current density, some neurosteroids, such as ALLO, SAGE-217, and SGE-516 modulated GABA receptor trafficking, while other neurosteroids, such as Ganaxolone did not modulate GABA receptor trafficking.

### Example 14: ALLO and P4 Increase S408/9 Phosphorylation in GT1-7 Cells

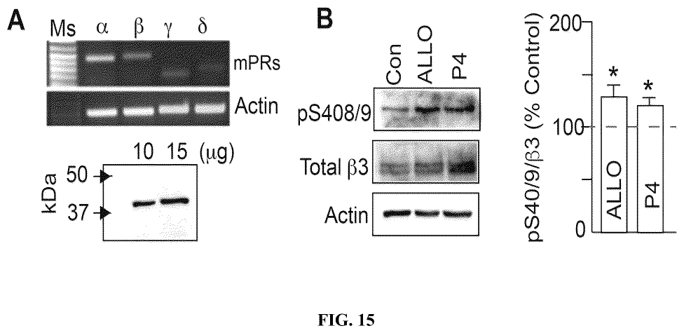

GT1-7 cells that co-express mPRα and GABA_{A}Rs were used to measure the level of phosphorylation at S408/9 position of 3 subunit. Quantitative PCR analysis indicated the enrichment of the mPRα mRNA in GT1-7 cells (FIG. 15A upper, figure taken from Thomas and Pang 2012). Expression of mPRα was confirmed using western blotting. 10 and 15 μg of SDS-soluble extracts from GT1-7 cells were immunoblotted with an mPRα specific antibody (FIG. 15A lower).

GT1-7 cells were treated with 100 nM AllO or P4 for 15 min and immunoblotted with antibodies specifically recognizing β3, actin, and phosphonate β3 subunit (pS408/9). The ratio of pS408/9 β3 subunit immunoreactivity were then normalized to levels seen in vehicle treated to controls, n=6. As shown in FIG. 15B, ALLO) and P4 increase S408/9 phosphorylation in GT1-7 cells.

### Example 15: Internal ALLO and ORG Induce Sustained Increases in GABA-Evoked Currents Recorded from GT1-7 Cells

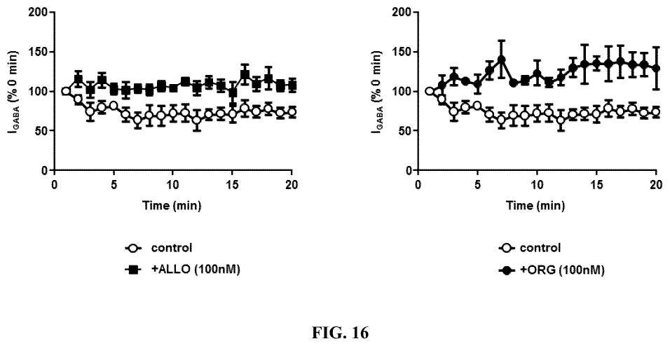

Patch-clamp recordings were made from GT1-7 cells. The magnitude of the GABA-induced currents (I_{GABA}) at EC20 agonists concentration were then measured, in the presence of control electrolyte or that supplemented with 100 nM ALLO, or 100 nM ORG OD 02-0 over a time course of 20 min. As shown in FIG. 16, ALLO and ORG OD 02-0 induced sustained increases in GABA-evoked currents recorded from GT1-7 cells.

### Example 16: ORG does not Acutely Modulate of the Function of GABA_{A}Rs Composed of α4β3 Subunits

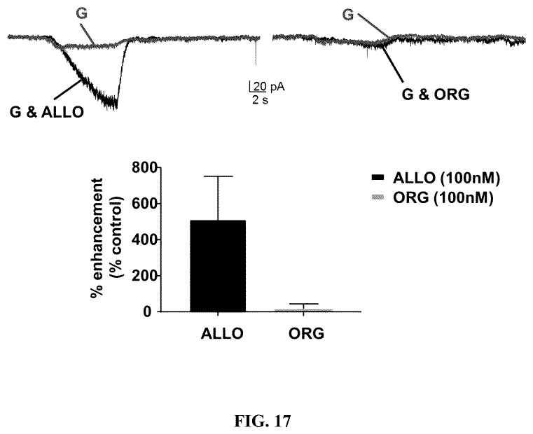

The magnitude of GABA-induced currents (I_{GABA}) was measured in HEK-293 expressing GABA_{A}Rs composed of al and β3 subunits. The effects of rapidly applied GABA (G), GABA and 100 nM ALLO (G&ALLO), or GABA and 100 mM ORG OD 02-0 (G&ORG) on GABA-evoked current (I_{GABA}) was then determined. Sample traces are shown in FIG. 17 upper panel. This data was then use to determine the potentiation of the GABA current by both drugs. As shown in FIG. 17 lower panel, ORG OD 02-0 compound did not acutely modulate of the function of GABA_{A}Rs composed of α4β3 subunits.

### Example 17: P4 and ORG-020 Regulates S408/9 Phosphorylation in Hippocampal Slices

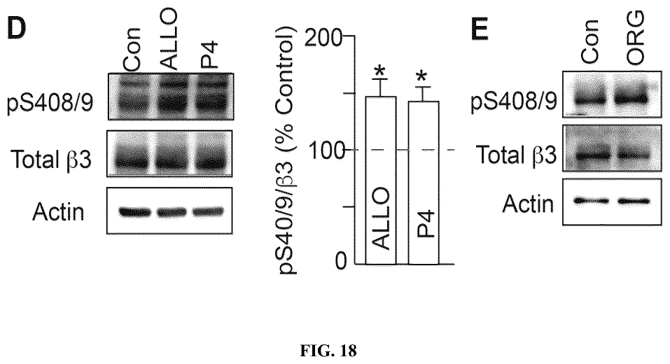

Hippocampal slices were treated with 100 nM ALLO or P4 (progesterone), and S408/9 phosphorylation was then determined as detailed above, n=4 slices (FIG. 18D). Hippocampal slices were treated with 100 nM ORG (FIG. 18E). S408/9 phosphorylation was examined using immunoblotting. In all panels; *=significantly different to control p<0.05 (one way ANOVA with Dunnet's multiple comparisons post-hoc test). As shown, P4 and ORG OD 02-0 regulated S408/9 phosphorylation in hippocampal slices.

### Example 18: ORG-02-0 and P4 Regulate a Tonic Current in Hippocampal Slices

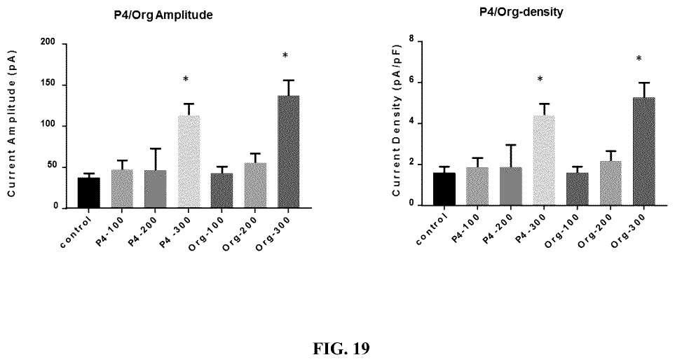

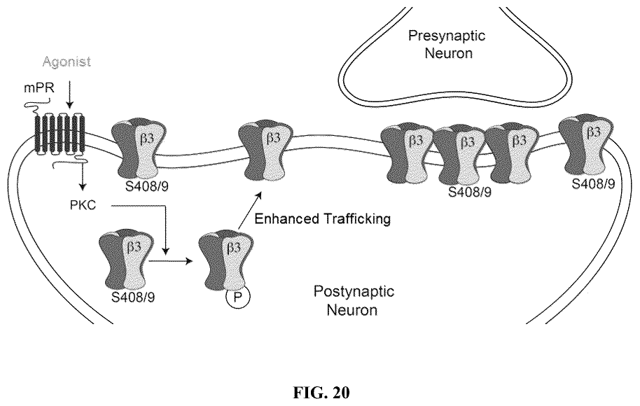

Dosage-dependent effect of P4 and ORG OD 02-0 compound in modulating GABAergic tonic current was measured. The ability of varying doses of P4 and ORG OD 02-0 to potentiate tonic current was determined as outlined in FIG. 3. The effects on current amplitude and density were then determined. *=significantly different to control p<0.05 (t-test n=6-8 cells). As shown in FIG. 19, both compounds exhibited dosage-dependent effect in modulating both the amplitude and density of GABAergic tonic current.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety.

The methods and processes illustratively described herein suitably may be practiced in differing orders of steps, and that they are not necessarily restricted to the orders of steps indicated herein or in the claims. Under no circumstances may the patent be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Other embodiments are within the following claims. In addition, where features or aspects of the invention are described in terms of Markush groups, those skilled in the art will recognize that the invention is also thereby described in terms of any individual member or subgroup of members of the Markush group.

### Embodiments of the Invention

1. A method for treating a CNS-related condition or disorder in a subject in need thereof, the method comprising administering to the subject a membrane progesterone receptor (mPR) agonist,
wherein the mPR agonist is not progesterone, 5α-DHP, allopregnanolone or testosterone.

2. The method of paragraph 1, wherein the mPR agonist is also a GABAergic modulator.

3. The method of paragraph 1, wherein the mPR agonist is not a GABAergic modulator.

4. A method for treating a CNS-related condition or disorder in a subject in need thereof, comprising administering to the subject
a) a membrane progesterone receptor (mPR) agonist; and
b) a GABAergic modulator.

5. The method of paragraph 1, 2 or 4, wherein the GABAergic modulator increases GABAergic inhibition in a cell through modulating intracellular trafficking of GABA receptors.

6. The method of paragraph 5, wherein the GABAergic modulator increases a membrane-associated amount of at least one GABA receptor subunit.

7. The method of paragraph 6, wherein the GABAergic modulator increases the membrane-associated amount of the at least one GABA receptor subunit by
(1) increasing an amount of the at least one GABA receptor subunit that is located on the cell membrane;
(2) increasing an amount of the at least one GABA receptor subunit that is incorporated into a GABA receptor;
(3) increasing an ratio between a membrane-associated amount of the at least one GABA receptor subunit and a soluble amount of the at least one GABA receptor subunit;
(4) reducing a rate of endocytosis of membrane GABA receptors, or any combination of (1)-(4).

8. The method of any one of paragraphs 1, 2 and 4-7, wherein the GABAergic modulator increases expression of at least one GABA receptor subunit in the cell.

9. The method of any one of paragraphs 1, 2 and 4-8, wherein the GABAergic modulator increases phosphorylation of at least one GABA receptor subunit in the cell.

10. The method of paragraph 9, wherein the phosphorylation is protein kinase C (PKC)-mediated phosphorylation.

11. The method of paragraph 8 or 9, wherein phosphorylation of an α4 GABA subunit is increased.

12. The method of any one of paragraphs 9-11, wherein phosphorylation of a β3 GABA subunit is increased.

13. The method of any one of paragraphs 9-12, wherein the phosphorylation occurs at S408/409 of the β3 subunit.

14. The method of any one of paragraphs 6-13, wherein the at least one GABA receptor subunit is selected from an al subunit, a β2 subunit, a γ2 subunit, an α4 subunit, a β3 subunit, and a δ subunit, and any combination thereof.

15. The method of any one of paragraphs 6-14, wherein the at least one GABA receptor subunit comprises a combination of α1β2γ2 subunits or a combination of α4β38 subunits.

16. The method of paragraph 5, wherein the GABA receptor is selected from a synaptic GABA receptor, an extrasynaptic GABA receptor, and a combination thereof.

17. The method of paragraph 16, wherein the synaptic GABA receptor comprises one or more subunits selected from an al subunit, a β2 subunit, and a γ2 subunit.

18. The method of paragraph 16 or 17, wherein the extrasynaptic GABA receptor comprises one or more subunits selected from an α4 subunit, a β3 subunit, and a δ subunit.

19. The method of paragraph 1, 2 or 4, wherein the GABAergic modulator increases GABAergic inhibition through potentiating GABA receptors in a cell.

20. The method of paragraph 19, wherein the GABAergic modulator increases the GABAergic current of the cell.

21. The method of paragraph 20, wherein the GABAergic current is a tonic current and/or a spontaneous inhibitory post-synpatic current (sIPSC).

22. The method of any one of paragraphs 19-21, wherein the GABAergic modulator increases
(1) an average amplitude of the tonic current;
(2) an average current density of the tonic current;
(3) an average amplitude of the sIPSC;
(4) an average decay time of the sIPSC, or
any combination of (1)-(4).

23. The method of any one of paragraphs 1-22, wherein the mPR agonist is a natural or synthetic neuroactive steroid.

24. The method of any one of paragraphs 1-23, wherein the mPR agonist is a progesterone analog.

25. The method of any one of paragraphs 5-24, wherein the GABA receptor is GABA_{A} receptor.

26. The method of any one of paragraphs 1-25, wherein the mPR agonist activates a mPR signaling pathway in a cell.

27. The method of any one of paragraphs 1-26, wherein upon activation of the mPR signaling pathway, protein kinase C (PKC) activity increases.

28. The method of any one of paragraphs 1-27, wherein upon activation of the mPR signaling pathway, the level of cellular cAMP reduces.

29. The method of any one of paragraphs 1-28, wherein upon activation of the mPR signaling pathway, the level of GABA-independent neural inhibition in the subject increases.

30. The method of any one of paragraphs 1-29, wherein the cell is a brain cell.

31. The method of any one of paragraphs 1-30, wherein the cell is a neuron.

32. The method of any one of paragraphs 1-31, wherein the CNS-related condition or disorder is a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, and/or a neurodegenerative disease and/or disorder.

33. The method of paragraph 32, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus.

34. The method of any one of paragraphs 1-33, wherein the mPR agonist and/or GABAergic modulator is administered in an amount sufficient to beneficially alter the CNS-related condition or disorder in said subject.

35. The method of any one of paragraphs 1-33, wherein the mPR agonist and/or GABAergic modulator is administered in an amount sufficient to beneficially alter brain excitability in said subject.

36. The method of any one of paragraphs 1-35, wherein the mPR agonist and/or the GABAergic modulator is administered orally, parenterally, intradermally, intrathecally, intramuscularly, subcutaneously, or transdermally.

37. A membrane progesterone receptor (mPR) agonist for use in treating a CNS-related condition or disorder in a subject, wherein the mPR agonist is not progesterone, 5α-DHP, allopregnanolone or testosterone.

38. The membrane progesterone receptor (mPR) agonist for use according to paragraph 37, wherein the mPR agonist is also a GABAergic modulator.

39. The membrane progesterone receptor (mPR) agonist for use according to paragraph 37, wherein the mPR agonist is not a GABAergic modulator.

40. A membrane progesterone receptor (mPR) agonist and a GABAergic modulator for use in treating a CNS-related condition or disorder in a subject.

41. The membrane progesterone receptor (mPR) agonist for use according to paragraph 37 or 38 or the membrane progesterone receptor (mPR) agonist and a GABAergic modulator for use according to paragraph 40, wherein the GABAergic modulator increases GABAergic inhibition in a cell through modulating intracellular trafficking of GABA receptors.

42. The membrane progesterone receptor (mPR) agonist for use or the membrane progesterone receptor (mPR) agonist and a GABAergic modulator for use according to paragraph 41, wherein the GABAergic modulator increases a membrane-associated amount of at least one GABA receptor subunit.

43. The membrane progesterone receptor (mPR) agonist for use or the membrane progesterone receptor (mPR) agonist and a GABAergic modulator for use according to paragraph 42, wherein the GABAergic modulator increases the membrane-associated amount of the at least one GABA receptor subunit by
(1) increasing an amount of the at least one GABA receptor subunit that is located on the cell membrane;
(2) increasing an amount of the at least one GABA receptor subunit that is incorporated into a GABA receptor;
(3) increasing an ratio between a membrane-associated amount of the at least one GABA receptor subunit and a soluble amount of the at least one GABA receptor subunit;
(4) reducing a rate of endocytosis of membrane GABA receptors, or
any combination of (1)-(4).

44. The membrane progesterone receptor (mPR) agonist for use according to paragraph 37 or 38 or the membrane progesterone receptor (mPR) agonist and a GABAergic modulator for use according to any one of paragraphs 40-43, wherein the GABAergic modulator increases expression of at least one GABA receptor subunit in the cell.

45. The membrane progesterone receptor (mPR) agonist for use according to paragraph 37 or 38 or the membrane progesterone receptor (mPR) agonist and a GABAergic modulator for use according to any one of paragraphs 40-44, wherein the GABAergic modulator increases phosphorylation of at least one GABA receptor subunit in the cell.

46. The membrane progesterone receptor (mPR) agonist for use or the membrane progesterone receptor (mPR) agonist and a GABAergic modulator for use according to paragraph 45, wherein the phosphorylation is protein kinase C (PKC)-mediated phosphorylation.

47. The membrane progesterone receptor (mPR) agonist for use or the membrane progesterone receptor (mPR) agonist and a GABAergic modulator for use according to paragraph 44 or 45, wherein phosphorylation of an α4 GABA subunit is increased.

48. The membrane progesterone receptor (mPR) agonist for use or the membrane progesterone receptor (mPR) agonist and a GABAergic modulator for use according to any one of paragraphs 45-47, wherein phosphorylation of a β3 GABA subunit is increased.

49. The membrane progesterone receptor (mPR) agonist for use or the membrane progesterone receptor (mPR) agonist and a GABAergic modulator for use according to any one of paragraphs 45-48, wherein the phosphorylation occurs at S408/409 of the β3 subunit.

50. The membrane progesterone receptor (mPR) agonist for use or the membrane progesterone receptor (mPR) agonist and a GABAergic modulator for use according to any one of paragraphs 42-49, wherein the at least one GABA receptor subunit is selected from an al subunit, a β2 subunit, a γ2 subunit, an α4 subunit, a β3 subunit, and a δ subunit, and any combination thereof.

51. The membrane progesterone receptor (mPR) agonist for use or the membrane progesterone receptor (mPR) agonist and a GABAergic modulator for use according to any one of paragraphs 42-50, wherein the at least one GABA receptor subunit comprises a combination of α1β2γ2 subunits or a combination of α4β3δ subunits.

52. The membrane progesterone receptor (mPR) agonist for use or the membrane progesterone receptor (mPR) agonist and a GABAergic modulator for use according to paragraph 41, wherein the GABA receptor is selected from a synaptic GABA receptor, an extrasynaptic GABA receptor, and a combination thereof.

53. The membrane progesterone receptor (mPR) agonist for use or the membrane progesterone receptor (mPR) agonist and a GABAergic modulator for use according to paragraph 52, wherein the synaptic GABA receptor comprises one or more subunits selected from an al subunit, a β2 subunit, and a γ2 subunit.

54. The membrane progesterone receptor (mPR) agonist for use or the membrane progesterone receptor (mPR) agonist and a GABAergic modulator for use according to paragraph 52 or 53, wherein the extrasynaptic GABA receptor comprises one or more subunits selected from an α4 subunit, a β3 subunit, and a δ subunit.

55. The membrane progesterone receptor (mPR) agonist for use according to paragraph 37 or 38 or the membrane progesterone receptor (mPR) agonist and a GABAergic modulator for use according paragraph 40, wherein the GABAergic modulator increases GABAergic inhibition through potentiating GABA receptors in a cell.

56. The membrane progesterone receptor (mPR) agonist for use or the membrane progesterone receptor (mPR) agonist and a GABAergic modulator for use according to paragraph 55, wherein the GABAergic modulator increases the GABAergic current of the cell.

57. The membrane progesterone receptor (mPR) agonist for use or the membrane progesterone receptor (mPR) agonist and a GABAergic modulator for use according to 56, wherein the GABAergic current is a tonic current and/or a spontaneous inhibitory post-synpatic current (sIPSC).

58. The membrane progesterone receptor (mPR) agonist for use or the membrane progesterone receptor (mPR) agonist and a GABAergic modulator for use according to any one of paragraphs 55-57, wherein the GABAergic modulator increases
(1) an average amplitude of the tonic current;
(2) an average current density of the tonic current;
(3) an average amplitude of the sIPSC;
(4) an average decay time of the sIPSC, or
any combination of (1)-(4).

59. The membrane progesterone receptor (mPR) agonist for use or the membrane progesterone receptor (mPR) agonist and a GABAergic modulator for use according to any one of paragraphs 37-58, wherein the mPR agonist is a natural or synthetic neuroactive steroid.

60. The membrane progesterone receptor (mPR) agonist for use or the membrane progesterone receptor (mPR) agonist and a GABAergic modulator for use according to any one of paragraphs 37-59, wherein the mPR agonist is a progesterone analog.

61. The membrane progesterone receptor (mPR) agonist for use or the membrane progesterone receptor (mPR) agonist and a GABAergic modulator for use according to any one of paragraphs 41-60, wherein the GABA receptor is GABA_{A} receptor.

62. The membrane progesterone receptor (mPR) agonist for use or the membrane progesterone receptor (mPR) agonist and a GABAergic modulator for use according to any one of paragraphs 37-61, wherein the mPR agonist activates a mPR signaling pathway in a cell.

63. The membrane progesterone receptor (mPR) agonist for use or the membrane progesterone receptor (mPR) agonist and a GABAergic modulator for use according to any one of paragraphs 37-62, wherein upon activation of the mPR signaling pathway, protein kinase C (PKC) activity increases.

64. The membrane progesterone receptor (mPR) agonist for use or the membrane progesterone receptor (mPR) agonist and a GABAergic modulator for use according to any one of paragraphs 37-63, wherein upon activation of the mPR signaling pathway, the level of cellular CAMP reduces.

65. The membrane progesterone receptor (mPR) agonist for use or the membrane progesterone receptor (mPR) agonist and a GABAergic modulator for use according to any one of paragraphs 37-64, wherein upon activation of the mPR signaling pathway, the level of GABA-independent neural inhibition in the subject increases.

66. The membrane progesterone receptor (mPR) agonist for use or the membrane progesterone receptor (mPR) agonist and a GABAergic modulator for use according to any one of paragraphs 37-65, wherein the cell is a brain cell.

67. The membrane progesterone receptor (mPR) agonist for use or the membrane progesterone receptor (mPR) agonist and a GABAergic modulator for use according to any one of paragraphs 37-66, wherein the cell is a neuron.

68. The membrane progesterone receptor (mPR) agonist for use or the membrane progesterone receptor (mPR) agonist and a GABAergic modulator for use according to any one of paragraphs 37-67, wherein the CNS-related condition or disorder is a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, and/or a neurodegenerative disease and/or disorder.

69. The membrane progesterone receptor (mPR) agonist for use or the membrane progesterone receptor (mPR) agonist and a GABAergic modulator for use according to paragraph 68, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus.

70. The membrane progesterone receptor (mPR) agonist for use or the membrane progesterone receptor (mPR) agonist and a GABAergic modulator for use according to any one of paragraphs 37-69, wherein the mPR agonist and/or GABAergic modulator is administered in an amount sufficient to beneficially alter the CNS-related condition or disorder in said subject.

71. The membrane progesterone receptor (mPR) agonist for use or the membrane progesterone receptor (mPR) agonist and a GABAergic modulator for use according to any one of paragraphs 37-69, wherein the mPR agonist and/or GABAergic modulator is administered in an amount sufficient to beneficially alter brain excitability in said subject.

72. The membrane progesterone receptor (mPR) agonist for use or the membrane progesterone receptor (mPR) agonist and a GABAergic modulator for use according to any one of paragraphs 37-71, wherein the mPR agonist and/or the GABAergic modulator is administered orally, parenterally, intradermally, intrathecally, intramuscularly, subcutaneously, or transdermally.

73. Use of a membrane progesterone receptor (mPR) agonist for the preparation of a medicament for treating a CNS-related condition or disorder in a subject, wherein the mPR agonist is not progesterone, 5α-DHP, allopregnanolone or testosterone.

74. The use according to paragraph 73, wherein the mPR agonist is also a GABAergic modulator.

75. The use according to paragraph 73, wherein the mPR agonist is not a GABAergic modulator.

76. Use of a membrane progesterone receptor (mPR) agonist and a GABAergic modulator for the preparation of a medicament for treating a CNS-related condition or disorder in a subject.

77. The use according to paragraphs 73, 74 or 76, wherein the GABAergic modulator increases GABAergic inhibition in a cell through modulating intracellular trafficking of GABA receptors.

78. The use according to paragraph 77, wherein the GABAergic modulator increases a membrane-associated amount of at least one GABA receptor subunit.

79. The use according to paragraph 78, wherein the GABAergic modulator increases the membrane-associated amount of the at least one GABA receptor subunit by
(1) increasing an amount of the at least one GABA receptor subunit that is located on the cell membrane;
(2) increasing an amount of the at least one GABA receptor subunit that is incorporated into a GABA receptor;
(3) increasing an ratio between a membrane-associated amount of the at least one GABA receptor subunit and a soluble amount of the at least one GABA receptor subunit;
(4) reducing a rate of endocytosis of membrane GABA receptors, or any combination of (1)-(4).

80. The use according to any one of paragraphs 73, 74 and 76-79, wherein the GABAergic modulator increases expression of at least one GABA receptor subunit in the cell.

81. The use according to any one of paragraphs 73, 74 and 76-80, wherein the GABAergic modulator increases phosphorylation of at least one GABA receptor subunit in the cell.

82. The use according to paragraph 81, wherein the phosphorylation is protein kinase C (PKC)-mediated phosphorylation.

83. The use according to paragraph 80 or 81, wherein phosphorylation of an α4 GABA subunit is increased.

84. The use according to any one of paragraphs 81-83, wherein phosphorylation of a β3 GABA subunit is increased.

85. The use according to any one of paragraphs 81-84, wherein the phosphorylation occurs at S408/409 of the β3 subunit.

86. The use according to any one of paragraphs 76-85, wherein the at least one GABA receptor subunit is selected from an al subunit, a β2 subunit, a γ2 subunit, an α4 subunit, a β3 subunit, and a δ subunit, and any combination thereof.

87. The use according to any one of paragraphs 78-86, wherein the at least one GABA receptor subunit comprises a combination of α1β2γ2 subunits or a combination of α4β38 subunits.

88. The use according to paragraph 77, wherein the GABA receptor is selected from a synaptic GABA receptor, an extrasynaptic GABA receptor, and a combination thereof.

89. The use according to paragraph 88, wherein the synaptic GABA receptor comprises one or more subunits selected from an al subunit, a β2 subunit, and a γ2 subunit.

90. The use according to paragraph 88 or 89, wherein the extrasynaptic GABA receptor comprises one or more subunits selected from an α4 subunit, a β3 subunit, and a 8 subunit.

91. The use according to paragraph 73, 74 or 67, wherein the GABAergic modulator increases GABAergic inhibition through potentiating GABA receptors in a cell.

92. The membrane progesterone receptor (mPR) agonist for use or the membrane progesterone receptor (mPR) agonist and a GABAergic modulator for use according to paragraph 91, wherein the GABAergic modulator increases the GABAergic current of the cell.

93. The use according to 92, wherein the GABAergic current is a tonic current and/or a spontaneous inhibitory post-synpatic current (sIPSC).

94. The use according to any one of paragraphs 91-93, wherein the GABAergic modulator increases
(1) an average amplitude of the tonic current;
(2) an average current density of the tonic current;
(3) an average amplitude of the sIPSC;
(4) an average decay time of the sIPSC, or
any combination of (1)-(4).

95. The use according to any one of paragraphs 73-94, wherein the mPR agonist is a natural or synthetic neuroactive steroid.

96. The use according to any one of paragraphs 73-95, wherein the mPR agonist is a progesterone analog.

97. The use according to any one of paragraphs 77-96, wherein the GABA receptor is GABA_{A} receptor.

98. The use according to any one of paragraphs 73-97, wherein the mPR agonist activates a mPR signaling pathway in a cell.

99. The use according to any one of paragraphs 73-98, wherein upon activation of the mPR signaling pathway, protein kinase C (PKC) activity increases.

100. The use according to any one of paragraphs 73-99, wherein upon activation of the mPR signaling pathway, the level of cellular cAMP reduces.

101. The use according to any one of paragraphs 73-100, wherein upon activation of the mPR signaling pathway, the level of GABA-independent neural inhibition in the subject increases.

102. The use according to any one of paragraphs 73-101, wherein the cell is a brain cell.

103. The use according to any one of paragraphs 73-102, wherein the cell is a neuron.

104. The use according to any one of paragraphs 73-103, wherein the CNS-related condition or disorder is a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, and/or a neurodegenerative disease and/or disorder.

105. The use according to paragraph 104, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus.

106. The use according to any one of paragraphs 73-105, wherein the mPR agonist and/or GABAergic modulator is administered in an amount sufficient to beneficially alter the CNS-related condition or disorder in said subject.

107. The use according to any one of paragraphs 73-106, wherein the mPR agonist and/or GABAergic modulator is administered in an amount sufficient to beneficially alter brain excitability in said subject.

108. The use according to any one of paragraphs 73-107, wherein the medicament is administered orally, parenterally, intradermally, intrathecally, intramuscularly, subcutaneously, or transdermally.

1. A method for treating a CNS-related condition or disorder in a subject in need thereof, the method comprising administering to the subject a membrane progesterone receptor (mPR) agonist, wherein the mPR agonist is not progesterone, 5α-DHP, allopregnanolone or testosterone.

2. The method of claim 1, wherein the mPR agonist is also a GABAergic modulator.

3. The method of claim 1, wherein the mPR agonist is not a GABAergic modulator.

4. A method for treating a CNS-related condition or disorder in a subject in need thereof, comprising administering to the subject
a) a membrane progesterone receptor (mPR) agonist; and
b) a GABAergic modulator.

5. The method of claim 2, wherein the GABAergic modulator increases GABAergic inhibition in a cell through modulating intracellular trafficking of GABA receptors.

6. The method of claim 5, wherein the GABAergic modulator increases a membrane-associated amount of at least one GABA receptor subunit.

7. The method of claim 6, wherein the GABAergic modulator increases the membrane-associated amount of the at least one GABA receptor subunit by
(1) increasing an amount of the at least one GABA receptor subunit that is located on the cell membrane;
(2) increasing an amount of the at least one GABA receptor subunit that is incorporated into a GABA receptor;
(3) increasing a ratio between a membrane-associated amount of the at least one GABA receptor subunit and a soluble amount of the at least one GABA receptor subunit;
(4) reducing a rate of endocytosis of membrane GABA receptors, or
any combination of (1)-(4).

8. The method of claim 2, wherein the GABAergic modulator increases expression of at least one GABA receptor subunit in a cell.

9. The method of claim 2, wherein the GABAergic modulator increases phosphorylation of at least one GABA receptor subunit in a cell.

10. The method of claim 9, wherein the phosphorylation is protein kinase C (PKC)-mediated phosphorylation.

11. The method of claim 9, wherein phosphorylation of an α4 GABA subunit is increased.

12. The method of claim 9, wherein phosphorylation of a β3 GABA subunit is increased.

13. The method of claim 9, wherein the phosphorylation occurs at S408/409 of the β3 subunit.

14. (canceled)

15. The method of claim 6, wherein the at least one GABA receptor subunit comprises a combination of α1β2γ2 subunits or a combination of α4β3δ subunits.

16. The method of claim 5, wherein the GABA receptor is selected from a synaptic GABA receptor, an extrasynaptic GABA receptor, and a combination thereof.

17. The method of claim 16, wherein the synaptic GABA receptor comprises one or more subunits selected from an al subunit, a β2 subunit, and a γ2 subunit.

18. The method of claim 16, wherein the extrasynaptic GABA receptor comprises one or more subunits selected from an α4 subunit, a β3 subunit, and a δ subunit.

19. The method of claim 1, wherein the GABAergic modulator increases GABAergic inhibition through potentiating GABA receptors in a cell.

20.-24. (canceled)

25. The method of claim 5, wherein the GABA receptor is a GABA_{A} receptor.

26.-31. (canceled)

32. The method of claim 1, wherein the CNS-related condition or disorder is a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, a neurodegenerative disease and/or disorder, a sleep disorder, a mood disorder, a schizophrenia spectrum disorder, a convulsive disorder, a disorder of memory and/or cognition, a movement disorder, a personality disorder, an autism spectrum disorder, pain, traumatic brain injury, a vascular disease, a substance abuse disorder and/or withdrawal syndrome, or tinnitus.

33.-72. (canceled)
